(19) United States
(12) Patent Application Publication
(10) Pub. No.: US 2026/0000715 A1
(43) Pub. Date: Jan. 1, 2026

(54) ROSEOMONAS MUCOSA, BACTERIAL FORMULATION AND EXOPOLYSACCHARIDE THEREOF, AND PREPARATION METHOD THEREFOR AND APPLICATION THEREOF

(71) Applicant: INSTITUTE OF DERMATOLOGY, CHINESE ACADEMY OF MEDICAL SCIENCES AND PEKING UNION MEDICAL COLLEGE, Nanjing (CN)

(72) Inventors: Qianjin LU, Nanjing (CN); Yijing ZHAN, Nanjing (CN); Siyu ZHOU, Nanjing (CN); Jinyu LIANG, Nanjing (CN)

(73) Assignee: INSTITUTE OF DERMATOLOGY, CHINESE ACADEMY OF MEDICAL SCIENCES AND PEKING UNION MEDICAL COLLEGE, Nanjing (CN)

(21) Appl. No.: 19/322,752

(22) Filed: Sep. 9, 2025

Related U.S. Application Data
(63) Continuation of application No. PCT/CN2024/080862, filed on Mar. 8, 2025.

(30) Foreign Application Priority Data
Mar. 10, 2023 (CN) 202310227440.X
Mar. 15, 2023 (CN) 202310247957.5
Mar. 22, 2023 (CN) 202310301745.0
May 17, 2023 (CN) 202310555890.1
May 18, 2023 (CN) 202310562830.2

(51) Int. Cl. A61K 35/74; A61K 8/73; A61K 8/99; A61K 31/715; A61P 17/06; A61P 17/16; A61P 17/18; A61P 29/00; A61P 31/04; A61Q 17/00; A61Q 19/00; A61Q 19/08; C12N 1/20; C12R 1/01

(52) U.S. Cl. CPC A61K 35/74; A61K 8/73; A61K 8/99; A61K 31/715; A61P 17/06; A61P 17/16; A61P 17/18; A61P 29/00; A61P 31/04; A61Q 17/005; A61Q 19/007; A61Q 19/08; C12N 1/205; A61K 2800/594; A61K 2800/805; A61K 2800/85; C12R 2001/01

(57) ABSTRACT

The invention discloses a Roseomonas mucosa, further discloses a bacterial formulation containing the Roseomonas mucosa above and an exopolysaccharide of the Roseomonas mucosa, and also discloses applications of the Roseomonas mucosa and the exopolysaccharide of the Roseomonas mucosa in prevention and/treatment of inflammation-related and/or T/B cell overactivation diseases, treatment or relief of cutaneous lupus erythematosus, treatment of psoriasis and atopic dermatitis, relief of UVB-induced skin damage, and preparation of a product for improving a skin condition. In the invention, a skin commensal bacteria strain is screened from skin of healthy people and has broad application prospects.

### CROSS-REFERENCE TO RELATED APPLICATIONS

This application is the U.S. continuation application of International Application No. PCT/CN2024/080862 filed on 8 Mar. 2024 which designated the U.S. and claims priority to Chinese Application Nos. CN 202310227440.X, CN 202310247957.5, CN 02310301745.0, CN 202310555890.1 and CN 202310562830.2 filed on 10 Mar. 2023, 15 Mar. 2023, 22 Mar. 2023, 17 May 2023 and 8 May 2023, respectively, the entire contents of each of which are hereby incorporated by reference.

### TECHNICAL FIELD

The present invention relates to the technical field of medicine, and particularly to a Roseomonas mucosa, a bacterial formulation containing the Roseomonas mucosa and an exopolysaccharide thereof, and a preparation method therefor and an application thereof.

### BACKGROUND

Autoimmune diseases refer to diseases in which an immune reaction of body to an autoantigen causes a tissue damage, which mainly comprise rheumatic arthritis, scleroderma, systemic lupus erythematosus, hyperthyroidism, ulcerative colitis, autoimmune hemolytic anemia, primary thrombocytopenic purpura, etc., affect about 5%-8% of the world's population, and bring great pain to patients. T and B lymphocyte immunomodulators may play an important role in the treatment of autoimmune diseases, such as inhibiting the proliferation and activation of T cells and regulating the balance of T cell subsets, inhibiting the proliferation, differentiation and antibody secretion of B cells, and other functions, which may become potentials for the immunomodulation treatment of autoimmune diseases. Because the abnormal activation of B cells plays an important role in many autoimmune diseases, a therapeutic drug targeting the B cells has always been a research hotspot. A classic B cell depleting agent CD20 monoclonal antibody has not only achieved a remarkable curative effect in systemic lupus erythematosus (SLE), but also achieved a considerable curative effect in rheumatoid arthritis, antineutrophil cytoplasmic antibody-related vasculitis and even Sjogren's syndrome recently.

A CD28 pathway is the most important co-stimulatory signal in an immune reaction response to an alloantigen, which may induce the activation of T cells, enhance a recognition ability of an immune system, lead to immune tolerance and reduce an immune response of body.

Lupus erythematosus is a classic autoimmune disease, which may cause damages to multiple organs, wherein patients suffering from a skin damage account for 70%-85%. Cutaneous lupus erythematosus (CLE) mainly causes the skin damage, and generally does not cause a visceral organ damage or causes a slight visceral organ damage. The CLE may be divided into acute cutaneous lupus erythematosus, subacute cutaneous lupus erythematosus and chronic cutaneous lupus erythematosus. Except the CLE, up to 90% of patients with the systemic lupus erythematosus have a skin manifestation, and 25-28% of the patients show a first symptom in skin. Clinically, some CLE subtypes may progress to the SLE, causing kidney and brain damages, thereby leading to renal failure and nervous system diseases. Therefore, the successful treatment of the CLE may significantly reduce the risk of systemic damage.

The CLE has complicated etiology and pathogenesis, which have not been completely clarified yet. Many studies show that genetic and environmental factors may cause the infiltration of immune cells, such as T cells, B cells, neutrophils, antigen presenting cells and NK cells, into a skin damage with CLE, and pro-inflammatory cytokines HMGB-1, interferon, IL-6, IL-17 and IL-12, and Th1 cell chemokines CXCL9, CXCL10, CXCL11, CXCL12, or the like, at the skin damage with CLE are significantly up-regulated. At present, therapeutic drugs comprise an antimalarial drug or a topical glucocorticoid, a calcineurin inhibitor, etc., but the drugs have poor curative effects on some patients, so that it is urgent to develop a safe and low-toxicity new therapy.

There are a large number of symbiotic bacterial floras on a skin surface, which play an important role in maintaining homeostasis of skin of a host and exerting a barrier function of skin, and may resist the invasion of pathogenic microorganisms, participate in an immune response of the host, maintain a normal structure of epidermis and regulate the repair and regeneration of the skin damage. Recent studies show that, according to phase I clinical trial results of skin symbiotic bacteria Staphylococcus hominis A9 (Sh A9) and R. mucosa in transplantation treatment of atopic dermatitis, this method is safe and effective, can improve the disease severity, and effectively avoids side effects of systemic drugs on an immune system of the host, thereby being a safe and low-toxicity new therapy.

Psoriasis is a common chronic T cell-mediated inflammatory skin disease, mainly characterized by abnormal proliferation and differentiation of epidermal keratinocytes, infiltration of immune cells and release of pro-inflammatory cytokines. Carbotriol (MC903) is an analogue of synthetic vitamin D3, which is widely used in the treatment of psoriasis and can inhibit the proliferation and differentiation of leukemia cells. In the psoriasis, immune cells, mainly T lymphocytes, infiltrate into local skin, and release cytokines, such as TNF-α, IFN-7 and IL-17, to induce an inflammatory reaction, which acts on the epidermal keratinocytes to make them proliferate excessively. M5 composed of five factors comprising IL-17A, IL-22, IL-1α, oncostatin M and TNF-α has been proved to induce the proliferation and inflammation of keratinocytes, which is a classic cell model of psoriasis.

A back part model constructed with imiquimod is a classic animal model of psoriasis, which is widely used in the study of psoriasis. After applying 62.5 mg of 5% imiquimod cream (Mikuimoterugao) to a back part for 6 consecutive days, a BALB/c mouse may be induced to gradually show typical psoriasis-like skin damages, such as scales, erythema, skin thickening and wrinkles, and histopathological changes, such as squamous epithelium and epidermal thickening, dyskeratosis and hyperkeratosis, dermal congestion and hemorrhage, and inflammatory cell infiltration, which are similar to manifestations of human psoriasis.

Inflammation is a mainly defensive immune response of the host to the stimulation of inflammatory factors, and many in-vivo cells are recruited into an immune microenvironment to produce cytokines, so as to eliminate the invasion of external factors and restore the body health. However, persistent inflammation may damage organs and systemic reactions of body, and plays an important role in the occurrence and development of many complex diseases.

Macrophages play an important role in regulating inflammatory response changes of the hose. Signals existing in different microenvironments from pathogens, injured tissues or activation effectors of adaptive immunity may trigger different genetic programs in differentiated macrophages, thereby inducing different functional polarization states. M1 and M2 are used to define pro-inflammation (in a case of activation with classical IFN-γ and lipopolysaccharide (LPS)) or anti-inflammation (in a case of activation with IL-4 or IL-10 respectively) in-vitro activation. M1-polarized macrophages are very important to trigger an inflammatory reaction after damaging, while M2 phenotypes seem to be very important to eliminate inflammation and regenerate damaged tissues. Macrophages in normal body often maintain a steady balance, but macrophage polarization imbalance may lead to the occurrence and development of many chronic inflammation-related diseases, such as sepsis, asthma, atherosclerosis and an inflammatory bowel disease. Therefore, inhibiting the persistent existence of M1-type macrophages and promoting the expression of M2-type phenotypes may benefit the inflammation-related diseases, and the polarization regulation of macrophages is expected to be a target of controlling and treating the inflammatory diseases.

Atopic dermatitis (AD) is a chronic and recurrent inflammatory skin disease mainly characterized by severe itching and eczema-like rash. Many factors, such as a genetic background, a skin barrier dysfunction, a regional immune abnormality and an environmental exposure, are jointly involved in the pathogenesis of the AD. In recent 30 years, a prevalence rate of the AD has increased rapidly, affecting as many as 20% of children and 10% of adults in developed countries. Skin flora imbalance may be one of the main environmental factors of the AD. Skin floras play an important role in skin barrier immune regulation, and resident bacteria on skin surface, such as Staphylococcus epidermidis, may regulate skin physiology and health by producing antimicrobial peptides and active metabolites. In recent years, more and more studies have shown that bacterial metabolites are important components of bacterial floras to play a biological role. Some bacterial exopolysaccharides may prevent the secretion of proinflammatory cytokines, revealing a new mechanism of immune response regulation with floras.

Carbotriol (MC903) is an analogue of synthetic vitamin D3, which is widely used in the treatment of psoriasis and can inhibit the proliferation and differentiation of leukemia cells. In recent years, local application of the MC903 can cause atopic dermatitis syndrome in a mouse, and at the same time, the MC903 can cause high expression of a thymic stromal lymphopoietin (TSLP) through a vitamin D receptor of epithelial keratinocytes. The TSLP is a cytokine derived from epithelial cells, which plays a key role in initiating and promoting Th2 cell-mediated allergic inflammation. In an acute phase of the AD, Th2 cytokines, such as IL-4, IL-5 and IL-10, are overexpressed, which stimulates high expression of IgE. Meanwhile, in a chronic phase of the AD, Th1 causes high expression of IFN-γ, IL-6 and TNF-α, and TNF-α is one of the well-known pro-inflammatory cytokines, so that the inhibition of the TNF-α is considered as a feasible strategy to prevent allergies.

With the increasing air pollution year by year, an ozone layer has been seriously damaged, and ultraviolet rays radiated to the earth's surface have increased significantly, affecting people's quality of life. UVB in sunlight may cause a series of skin diseases, such as sunburn, hyperpigmentation and skin squamous cell carcinoma. Skin keratinocytes (HaCaT) are main cells forming the epidermis, and the HaCaT cells are main targets of the UVB on the epidermis. Almost all UVB is absorbed by the HaCaT cells, which may induce the HaCaT cells to produce excessive reactive oxygen species (ROS), destroy their own antioxidant defense system, cause lipid peroxidation, affect related signal transduction pathways, and damage cell structure or function. UVB radiation may induce the secretion of interleukin-6 (IL-6), interleukin-13 (IL-13) and other inflammatory factors to mediate an inflammatory reaction, regulate an immune response, and induce apoptosis and other cellular physiological activities, thereby causing adverse manifestations of skin, such as erythema, red and swollen, laxity, dryness, desquamation and hyperpigmentation.

As the largest organ of human body, skin is a first protective barrier between internal organs and external environment, and a first defense line of human body against physical, chemical, biological and other external pathogenic factors. With the continuous improvement of living standards, people pay more and more attention to skin problems caused by skin dryness and aging, and skin care products or drugs with related effects are often used for skin care.

Skin microecology of human body is an ecosystem composed of microorganisms, such as bacteria, fungi, viruses and mites, skin tissues, cell secretions, etc. Resident floras of skin participate in skin sebum metabolism, maintain a pH value of skin surface and decompose cutin, thereby playing a role of biological barrier for skin to resist the invasion of external pathogens. Metabolites of skin probiotics have direct or indirect protective effects on the host, and can improve the diversity of beneficial bacterial strains on skin surface, thereby improving the skin condition.

A skin commensal bacteria strain screened from skin of healthy people and a secretion exopolysaccharide thereof have broad application prospects.

### SUMMARY

Objective of the invention: the present invention provides a Roseomonas mucosa screened from skin of healthy people and a secretion exopolysaccharide thereof, and further provides an application thereof.

The present invention further provides a live bacterial formulation of a Roseomonas mucosa and a preparation method for an exopolysaccharide of the Roseomonas mucosa, and the present invention further provides an application of the live bacterial formulation of the Roseomonas mucosa and the exopolysaccharide of the Roseomonas mucosa in relieving cutaneous lupus erythematosus, treating psoriasis and atopic dermatitis, and relieving a UVB-induced skin damage, and in preparing a product for improving a skin condition.

In order to solve the above technical problems, the present invention discloses a Roseomonas mucosa, which is classified and named as Roseomonas mucosa, with a strain number of DL-1, and is already preserved in China General Microbiological Culture Collection Center, with a preservation date of Oct. 26, 2022, a preservation number of CGMCC No. 100101, and a preservation address of Collection Center of Institute of Microbiology, Chinese Academy of Sciences, No. 3, Yard 1, Beichen West Road, Chaoyang District, Beijing, postcode 100101. This bacterial strain is a skin commensal bacteria strain screened from skin of healthy people by the inventor in November, 2021.

The Roseomonas mucosa DL-1 has the following properties:

### Morphological Characteristics of Bacterial Colony

After being cultured in an R2A medium at 32° C. for 16-24 hours, it is observed through a microscope that the bacterial colony is a single bacterial colony, with a morphology as shown in FIG. 1A. The Roseomonas mucosa DL-1 is classified to Acetobacteraceae in Rhodospirillales and coccobacillus, belongs to a Gram-negative bacterium, produces red pigment, is mucinous in shape and arranged in single, paired or short-chain shaped, and has flagella, thereby being able to move. After being cultured in the above medium at 32° C. for 12 hours, the bacteria cells may grow in large quantities, wherein a temperature range of growth is 5-40° C., optimally 30-35° C., and a pH range of growth is 5.0-9.5, optimally 6.5-7.5. The bacteria cells may grow normally in LB, R2A, TSB, BAB and other media.

Analysis of 16S rDNA Sequence

A length of the 16S rDNA sequence is 1464 bp. Comparing the 16S rDNA sequence with related species in a GeneBank database, a similarity between the bacterial strain DL-1 and the Roseomonas mucosa reaches 99.86%. Therefore, the bacterial strain of the present invention is identified as the Roseomonas mucosa, with a strain number of DL-1, and is preserved in China General Microbiological Culture Collection Center, with a preservation date of Oct. 26, 2022 and a preservation number of CGMCC 25967.

The present invention further provides a live bacterial formulation of a Roseomonas mucosa, wherein the live bacterial formulation contains the Roseomonas mucosa DL-1 (R. mucosa DL-1) above.

A viable count of the Roseomonas mucosa in the live bacterial formulation is 1×10⁴-9×10¹¹ CFU/mL, preferably 1×10⁶-1×10⁹ CFU/mL.

The present invention further provides a preparation method for the live bacterial formulation of the Roseomonas mucosa above, wherein the Roseomonas mucosa is inoculated in an R2A medium to activate at 25-37° C. for 12-24 hours, and the activated Roseomonas mucosa is transferred to a fermentation medium to culture at 30-37° C. for 24-36 hours, and then added with a vehicle to obtain the, wherein the vehicle is any one of glycerol and propylene glycol.

The R2A medium contains ingredients of: 0.5 g/L yeast extract powder, 0.5 g/L peptone, 0.5 g/L casein hydrolysate, 0.5 g/L glucose, 0.5 g/L soluble starch, 0.3 g/L dipotassium hydrogen phosphate, 0.024 g/L anhydrous magnesium sulfate, 0.3 g/L sodium pyruvate and 15.0 g/L agar, and has a pH value of 7.2.

Preferably, the fermentation medium has a formula as follows: 10-100 g/L carbon source, 1-30 g/L nitrogen source and 0.01-50 g/L inorganic salt, and has a pH value of 5.0-9.0, and the solvent is water.

The carbon source is any one or a combination of several of glucose, sucrose, maltose, lactose, xylose, fructose, lactic acid, citric acid, glycerol, starch and molasses, preferably any one or a combination of several of sucrose, glucose, lactose, citric acid and starch; the nitrogen source is any one or a combination of several of yeast extract powder, beef extract, peptone, yeast extract, corn steep liquor, soybean cake powder, cottonseed cake powder, urea, (NH₄)₂SO₄, NH₄Cl, (NH₄)₂HPO₄ and NH₄NO₃, preferably any one or a combination of several of peptone, yeast extract powder, corn steep liquor, (NH₄)₂SO4, NH₄Cl, (NH₄)₂HPO₄ and NH₄NO₃; and the inorganic salt is any one or a combination of several of sodium chloride, sulfate, phosphate, dihydrogen phosphate, hydrogen phosphate and hydrochloride.

Preferably, the fermentation medium is an enriched medium rich in the carbon source and the nitrogen source, such as:
a TSB (EP) fermentation medium 1: 17.0 g/L tryptone, 3.0 g/L soybean peptone, 5.0 g/L sodium chloride, 2.5 g/L dipotassium hydrogen phosphate and 2.5 g/L glucose, at a pH value of 7.3;
a TSB (Pharmacopoeia of the People's Republic of China) fermentation medium 2: 17 g/L tryptone, 5 g/L sodium chloride, 3 g/L soybean papain hydrolysate, 2.5 g/L dipotassium hydrogen phosphate and 2.5 g/L glucose, at a pH value of 7.3; and
an LB fermentation medium: 10 g/L tryptone, 5 g/L yeast extract powder and 10 g/L sodium chloride, at a pH value of 7.0.

In a specific embodiment, the fermentation medium contains ingredients of: 17.0 g/L tryptone, 3.0 g/L soybean peptone, 5.0 g/L sodium chloride, 2.5 g/L dipotassium hydrogen phosphate and 2.5 g/L glucose, at a pH value of 7.3.

The present invention further provides an exopolysaccharide of a Roseomonas mucosa, which is obtained by sterilization, deproteinization, precipitation with absolute ethanol, and separation purification with DEAE centrifugal exchange resin of a fermentation broth containing the Roseomonas mucosa.

Specifically, the fermentation broth is centrifuged at 3,000-5,000 rpm for 10 minutes for sterilization, added with 1/2-fold volume of sevage reagent for deproteinization for 3 times, and then added with 1-fold volume of absolute ethanol for precipitation at 4° C. overnight to obtain a crude exopolysaccharide precipitate, which is redissolved with deionized water, purified by DEAE-52, and then freeze-dried to obtain the exopolysaccharide (R. mucosa DL-1 EPS, EPS).

A weight-average molecular weight of the exopolysaccharide of the Roseomonas mucosa is 3,000-3,500 Da, and a monosaccharide composition comprises arabinose, rhamnose, galactose, glucose, xylose, mannose, ribose, galacturonic acid, glucuronic acid, mannuronic acid and guluronic acid.

The monosaccharide composition of the exopolysaccharide of the Roseomonas mucosa is: arabinose:rhamnose:galactose:glucose:xylose:mannose:ribose:galacturonic acid:glucuronic acid:mannuronic acid:guluronic acid=0.50-0.6:1.3-1.5:1.3-2:72-78:4-7:9-11:0.9-1.1:0.5-2.4:0.6-1.4:0.6-1.4:0.9-1.7.

In a specific embodiment, the weight-average molecular weight of the exopolysaccharide is 3,000 Da, and the monosaccharide composition is arabinose:rhamnose:galactose:glucose:xylose:mannose:ribose:galacturonic acid:glucuronic acid:mannuronic acid:guluronic acid=0.51:1.47:1.8:73.98:4.67:9.96:0.94:2.33:1.35:1.32:1.67.

The present invention further provides an application of the exopolysaccharide of the Roseomonas mucosa in preparation of a drug for preventing and/or treating inflammation-related and or T/B cell overactivation diseases.

In one aspect, it is found in the present application that the exopolysaccharide of the Roseomonas mucosa inhibits differentiation of B cells into plasmablasts and antibody secreting cells induced in vitro. Specifically, 25 μg/mL-75 μg/mL exopolysaccharide of the Roseomonas mucosa DL-1 may inhibit differentiation of B cells into plasmablasts induced by 30 ng/mL IL-4 and 100 ng/mL R848 in vitro.

Meanwhile, the exopolysaccharide of the Roseomonas mucosa inhibits activation of T cells into Tfh cells induced by anti-CD28 in vitro. Specifically, 5 μg/mL-500 μg/mL exopolysaccharide of the Roseomonas mucosa DL-1 may inhibit activation of T cells into Tfh cells induced by 5 μg/mL anti-CD3 and 2 μg/mL anti-CD28 in vitro.

The present invention further provides an application of the exopolysaccharide of the Roseomonas mucosa above in preparation of a drug or a formulation for treating a cutaneous lupus erythematosus skin damage.

In application, the live bacterial formulation or the exopolysaccharide is locally and topically applied to a skin damage of a mouse with cutaneous lupus erythematosus induced by UVB and Pristane. The live bacterial formulation may be used together with a drug in diagnosis and treatment consensus of cutaneous lupus erythematosus, or used alone to regulate an immune microenvironment at the skin damage, thereby reducing deposition of IgG immune complexes, relieving the skin damage and reducing a dosage of a clinical glucocorticoid.

Preferably, a count of the live R. mucosa DL-1 in the live bacterial formulation of the R. mucosa DL-1 applied locally and topically is 1×10⁷-1×10⁹ CFU/mL.

Preferably, the live bacterial formulation of the R. mucosa DL-1 applied locally and topically starts to be applied after UVB irradiation, wherein a dosage of the live bacterial formulation in local and topical application is 1×10⁷-1×10⁹ CFU/cm², and the live bacterial formulation is applied once a day.

The exopolysaccharide of the R. mucosa DL-1 applied locally and topically starts to be applied after UVB irradiation, wherein a dosage of the exopolysaccharide in local and topical application is 0.1 mg/cm²-0.3 mg/cm², and the exopolysaccharide is applied once a day.

The cutaneous lupus erythematosus skin damage comprises one or more of acute cutaneous lupus erythematosus, subacute cutaneous lupus erythematosus, chronic cutaneous lupus erythematosus and subtypes thereof.

Studies of the present application show that the live bacterial formulation or the exopolysaccharide of the Roseomonas mucosa regulates an immune microenvironment at the skin damage, thereby reducing deposition of IgG immune complexes.

It is also found in the present application that the live bacterial formulation of the Roseomonas mucosa relieves a psoriasis-like skin damage on a back part of a BALB/c mouse induced by imiquimod, inhibits an increase of a spleen index of the BALB/c mouse with psoriasis induced by imiquimod, reduces an imiquimod-induced psoriasis-like skin damage, and reduces epidermal thickness of the BALB/c mouse induced by imiquimod.

The present invention further provides a function of the exopolysaccharide DL-EPS of the R. mucosa DL-1 in relieving a psoriasis cell model.

Specifically, in an in-vitro experiment, the exopolysaccharide of the Roseomonas mucosa inhibits proliferation of HaCaT cells induced by M5. More specifically, the exopolysaccharide of the Roseomonas mucosa DL-1 may inhibit the proliferation of the HaCaT cells induced by 10 ng/mL M5 by using a 10 ng/mL M5-induced HaCaT cell proliferation and inflammation model.

The exopolysaccharide of the Roseomonas mucosa inhibits an increase of the mRNA expression of an inflammatory factor of the HaCaT cells induced by M5. Specifically, the exopolysaccharide of the Roseomonas mucosa DL-1 may inhibit the increase of the transcription level of the inflammatory factor of the HaCaT cells induced by 10 ng/mL M5.

The exopolysaccharide of the Roseomonas mucosa relieves a skin damage on a back part of a BALB/c mouse with psoriasis induced by imiquimod. Specifically, 62.5 mg of imiquimod is evenly applied to a back part of BALB/c mice in each group respectively to induce for 6 consecutive days. Meanwhile, the mice are divided into a control group, a matrix group, a carbotriol group, a live bacterial formulation group, a high-dose polysaccharide group and a low-dose polysaccharide group the experiment, administered in the morning, and modeled in the afternoon, and the mice are sacrificed for 6 consecutive days to observe the efficacy. Results show that the exopolysaccharide of the Roseomonas mucosa DL-1 may relieve skin erythema, scales and thickening of the BALB/c mouse induced by imiquimod.

The exopolysaccharide of the Roseomonas mucosa reduces the spleen index of the BALB/c mouse with psoriasis induced by imiquimod.

Research results of the present application show that the exopolysaccharide of the Roseomonas mucosa DL-1 regulates polarization of macrophages Raw 264.7 induced by lipopolysaccharide, inhibits M1-type macrophages, and promotes M2-type macrophages.

In another aspect, the exopolysaccharide of the Roseomonas mucosa DL-1 inhibits secretion of an inflammatory factor of macrophages Raw 264.7 induced by lipopolysaccharide, or lipopolysaccharide and IFN-7.

The present invention further provides a function of the exopolysaccharide EPS of the R. mucosa DL-1 in relieving an atopic dermatitis cell model.

Preferably, in an in-vitro experiment, an AD cell model is formed by HaCaT cells induced by 10 ng/mL TNF-α and 10 ng/mL IFN-7, and the exopolysaccharide of the Roseomonas mucosa DL-1 may inhibit an increase of the mRNA expression of an inflammatory factor of the HaCaT cells induced by 10 ng/mL TNF-α and 10 ng/mL IFN-7.

Preferably, the exopolysaccharide of the Roseomonas mucosa DL-1 may inhibit increases of transcription levels of TSLP, IL-33, TNF-α, IL-6 and IL-10 of the HaCaT cells induced by 10 ng/mL TNF-α and 10 ng/mL IFN-7.

The present invention further provides a function of the exopolysaccharide EPS of the R. mucosa DL-1 in relieving an atopic dermatitis animal model.

Specifically, 1 nmol MC903 is applied to a left ear of BALB/c mice in each group respectively to induce for 15 consecutive days. Meanwhile, the mice are divided into a control group, a matrix group, a mometasone furoate group, a high-dose polysaccharide group and a low-dose polysaccharide group the experiment, administered in the morning, and modeled in the afternoon, and the mice are sacrificed for 15 consecutive days to observe the efficacy.

Preferably, the exopolysaccharide of the Roseomonas mucosa DL-1 may relieve a skin damage of a BALB/c mouse with atopic dermatitis induced by MC903, comprising ear swelling and scales.

Preferably, the exopolysaccharide of the Roseomonas mucosa DL-1 may relieve ear thickening of the BALB/c mouse induced by MC903.

Preferably, the exopolysaccharide of the Roseomonas mucosa DL-1 may relieve mast cell infiltration of the BALB/c mouse induced by MC903.

Preferably, the exopolysaccharide of the Roseomonas mucosa DL-1 may inhibit increases of transcription levels of inflammatory factors, such as IL-6, IL-1β, TNF-α, TSLP and IL-33, at the skin damage of the BALB/c mouse induced by MC903.

Preferably, the exopolysaccharide of the Roseomonas mucosa may inhibit a decrease of a filaggrin expression at the skin damage of the BALB/c mouse induced by MC903.

The present invention further provides a function of the live bacterial formulation of the skin symbiotic bacterium Roseomonas mucosa and the exopolysaccharide of the Roseomonas mucosa in relieving transcription level detection of inflammatory factors in HaCaT cells and an acute back damage of a mouse induced by UVB, and a pathophysiological state.

A count of the live Roseomonas mucosa in the live bacterial formulation is 1×10⁶-1×10⁹ CFU/mL.

Preferably, the count of the live Roseomonas mucosa is 1×10⁵-1×10⁷ CFU/mL; and more preferably, the viable count of the Roseomonas mucosa is 1×10⁵-1×10⁶ CFU/mL.

Research results show that the live bacterial formulation of the Roseomonas mucosa reduces the level of oxidative stress and apoptosis of HaCaT cells induced by UVB irradiation.

The viable bacteria formulation of the Roseomonas mucosa improves a skin damage on a back part of a C57BL/6 mouse induced by UVB irradiation, comprising erythema, edema, exudation and scabbing, inhibits apoptosis of keratinocytes at a skin damage on a back part induced by UVB irradiation, and reduces the mRNA expression of an inflammatory factor at the skin damage.

The present invention further provides an application of the exopolysaccharide of the Roseomonas mucosa in preparation of a drug for relieving a UVB-induced skin damage. It is found from studies in the present application that the exopolysaccharide of the Roseomonas mucosa also has a good application prospect in relieving related diseases induced by UVB, photoaging and after-sun repair.

Specifically, the exopolysaccharide of the Roseomonas mucosa reduces oxidative stress and apoptosis of HaCaT cells induced by UVB irradiation, and reduces the mRNA expression of an inflammatory factor of the HaCaT cells induced by UVB irradiation; the exopolysaccharide of the Roseomonas mucosa inhibits an increase of the mRNA expression of a type I interferon IFN-β induced by UVB irradiation; and the exopolysaccharide of the Roseomonas mucosa improves skin erythema, edema, exudation and scabbing on the back part of the C57BL/6 mouse induced by UVB irradiation, inhibits apoptosis of keratinocytes at a skin damage on a back part induced by UVB irradiation, and reduces the mRNA expression of an inflammatory factor at the skin damage.

Specifically, a dosage of the exopolysaccharide DL-EPS in a cell model is 0.5-4.5 mg/mL, and a dosage of the exopolysaccharide DL-EPS in local and topical treatment for a UVB-induced C57/BL6 acute model is 0.1-0.5 mg/cm².

The live bacterial formulation of the Roseomonas mucosa and the exopolysaccharide of the Roseomonas mucosa may be used together with an antibiotic-free drug and a skin care product, or used alone.

Beneficial effects: according to the present invention, the skin symbiotic bacterium R. mucosa DL-1 with an environmental cutaneous lupus skin damage is screened from healthy people, the R. mucosa DL-1 is used as a bacterial strain for fermentation culture and then separated to obtain the live bacterial formulation of the R. mucosa DL-1 and the exopolysaccharide of the R. mucosa DL-1, and the R. mucosa DL-1 is colonized on the epidermis of the skin damage to regulate the immune microenvironment of the skin damage, thereby reducing the deposition of the IgG immune complexes, relieving the skin damage and reducing the dosage of the clinical glucocorticoid. The live bacterial formulation of the R. mucosa DL-1 and the exopolysaccharide of the R. mucosa DL-1 provided by the present invention have the following effects:
(1) the live bacterial formulation of the R. mucosa DL-1 and the exopolysaccharide of the R. mucosa DL-1 provided by the present invention are safe and effective in treating the cutaneous lupus erythematosus skin damage, without side effects on the skin or the system, thereby having good application prospects, and the live bacterial formulation of the symbiotic bacterium and the metabolite exopolysaccharide of the symbiotic bacterium are applied to relieve the skin damage and local inflammation of immune-related skin diseases, thereby being of great significance to the application and development of a skin microbiome in the immune-related skin diseases;
(2) the exopolysaccharide of the Roseomonas mucosa DL-1 provided by the present invention may inhibit the proliferation of the HaCaT cells and the secretion of the inflammatory factor induced by M5, and the imiquimod-induced BALB/c psoriasis animal model verifies the relieving effect of the Roseomonas mucosa DL-1 and the exopolysaccharide thereof, so that the Roseomonas mucosa DL-1 and the exopolysaccharide thereof have certain application prospects in inflammatory skin diseases;
(3) the exopolysaccharide of the Roseomonas mucosa DL-1 provided by the present invention may inhibit the secretion of the inflammatory factor of the HaCaT cells induced by 10 ng/mL TNF-α and 10 ng/mL IFN-γ; inhibit the secretion of TSLP, IL-33, TNF-α, IL-6 and IL-1β of the HaCaT cells induced by 10 ng/mL TNF-α and 10 ng/mL IFN-γ; and relieve an atopic dermatitis skin damage of the BALB/c mouse induced by MC903;
(4) the live bacterial formulation of the R. mucosa DL-1 and the exopolysaccharide DL-EPS provided by the present application may resist the oxidative stress of the HaCaT cells induced by UVB irradiation, reduce the transcription level of the inflammatory factor of the HaCaT cells induced by 50 mJ/cm² UVB at the same time, reduce the apoptosis of the HaCaT cells induced by 50 mJ/cm² UVB, effectively relieve pathological changes of a skin damage visible to the naked eye and a skin damage under the microscope of the C57BL/6 mouse induced by 430 mJ/cm² UVB, and inhibit transcription of important inflammatory factors IL-1, IL-6 and TNF—at the skin damage of the C57BL/6 mouse induced by 430 mJ/cm² UVB; and the live bacterial formulation of the skin symbiotic bacterium R. mucosa and the exopolysaccharide of the skin symbiotic bacterium in the present invention have a certain protective effect on the apoptosis of the epidermal keratinocytes induced by UVB, thereby improving UVB-induced acute skin inflammation; and
(5) the Roseomonas mucosa and the exopolysaccharide thereof provided by the present application have excellent effects in improvement of skin barrier, moisturization, enhancement of skin antimicrobial defense, renewal of aged stratum corneum and anti-aging, thereby being suitable for preparing foods, drugs, cosmetics and the like for improving skin-related functions.

### BRIEF DESCRIPTION OF THE DRAWINGS

FIG. 1A shows biological characteristics of a Roseomonas mucosa: colony morphology;

FIG. 1B shows biological characteristics of a Roseomonas mucosa: Gram staining;

FIG. 1C shows biological characteristics of a Roseomonas mucosa: scanning electron micrograph;

FIG. 1D shows biological characteristics of a Roseomonas mucosa: phylogenetic tree analysis of 16S rRNA sequence;

FIG. 2 shows an elution curve of an exopolysaccharide EPS of a Roseomonas mucosa DL-1;

FIG. 3 is an ion chromatogram of a standard sample;

FIG. 4 is an ion chromatogram of an exopolysaccharide EPS sample;

FIG. 5 is a scattered spectrogram of the exopolysaccharide EPS sample;

FIG. 6 shows an influence of the exopolysaccharide EPS of the Roseomonas mucosa DL-1 on differentiation of B cells induced in vitro;

FIG. 7 shows an influence of the exopolysaccharide EPS of the Roseomonas mucosa DL-1 on activation of T cells;

FIG. 8 shows an influence of a live bacterial formulation of the skin symbiotic bacterium R. mucosa DL-1 on a CLE skin damage of C57BL/6J mice induced by a combination of Pristane and UVB and a pathology of the skin damage;

FIG. 9 shows an influence of the live bacterial formulation of the skin symbiotic bacterium R. mucosa DL-1 on deposition of an immune complex at the CLE skin damage of the C57BL/6J mice induced by the combination of Pristane and UVB;

FIG. 10 shows an influence of the live bacterial formulation of the skin symbiotic bacterium R. mucosa DL-1 on local immune cells at the CLE skin damage of the C57BL/6J mice induced by the combination of Pristane and UVB;

FIG. 11 shows an influence of the exopolysaccharide EPS of the skin symbiotic bacterium R. mucosa DL-1 on the CLE skin damage of the C57BL/6J mice induced by the combination of Pristane and UVB and the pathology of the skin damage;

FIG. 12 shows an influence of the exopolysaccharide EPS of the skin symbiotic bacterium R. mucosa DL-1 on the deposition of the immune complex at the CLE skin damage of the C57BL/6J mice induced by the combination of Pristane and UVB;

FIG. 13 shows an influence of the exopolysaccharide EPS of the skin symbiotic bacterium R. mucosa DL-1 on transcription levels of inflammatory factors IL-6, IL-13 and IL-17A at the CLE skin damage of the C57BL/6J mice induced by the combination of Pristane and UVB;

FIG. 14 shows a toxic effect of an exopolysaccharide DL-EPS of the Roseomonas mucosa DL-1 on HaCaT cells;

FIG. 15 shows an influence of the exopolysaccharide DL-EPS of the Roseomonas mucosa DL-1 on proliferation of the HaCaT cells induced by M5;

FIG. 16 shows an influence of the exopolysaccharide DL-EPS of the Roseomonas mucosa DL-Ion the mRNA expression of an inflammatory factor of the HaCaT cells induced by M5;

FIG. 17 shows an influence of the exopolysaccharide DL-EPS of the Roseomonas mucosa DL-1 on a psoriasis skin damage of BALB/c mice induced by imiquimod;

FIG. 18 shows an influence of the exopolysaccharide DL-EPS of the Roseomonas mucosa DL-1 on PASI scoring of the psoriasis skin damage of the BALB/c mice induced by imiquimod;

FIG. 19 shows an influence of the exopolysaccharide DL-EPS of the Roseomonas mucosa DL-1 on a spleen index of the BALB/c mice induced by imiquimod;

FIG. 20 shows an influence of the exopolysaccharide DL-EPS of the Roseomonas mucosa DL-1 on a pathology of the psoriasis-like skin damage of the BALB/c mice induced by imiquimod;

FIG. 21 shows an influence of the live bacterial formulation of the Roseomonas mucosa R. mucosa DL-1 and the exopolysaccharide DL-EPS of the Roseomonas mucosa R. mucosa DL-1 on epidermal thickness of the psoriasis-like skin damage of the BALB/c mice induced by imiquimod;

FIG. 22 shows a toxic effect of the exopolysaccharide EPS of the Roseomonas mucosa DL-1 on Raw 264.7 cells;

FIG. 23 shows an influence of the exopolysaccharide EPS of the Roseomonas mucosa DL-1 on the mRNA expression of an inflammatory factor of the Raw 264.7 cells induced by lipopolysaccharide; FIG. A-B shows treatment with the lipopolysaccharide for 4 hours, and FIG. C-D shows treatment with the lipopolysaccharide for 24 hours;

FIG. 24 shows an influence of the exopolysaccharide EPS of the Roseomonas mucosa DL-1 on polarization of the Raw 264.7 cells induced by lipopolysaccharide;

FIG. 25 shows an influence of the exopolysaccharide EPS of the Roseomonas mucosa DL-1 on the mRNA expression of an inflammatory factor of the Raw 264.7 cells induced by lipopolysaccharide and IFN-7;

FIG. 26 shows an influence of the exopolysaccharide EPS of the Roseomonas mucosa DL-1 on a viability of the HaCaT cells;

FIG. 27 shows an influence of the exopolysaccharide EPS of the Roseomonas mucosa DL-1 on the mRNA expression of an inflammatory factor of the HaCaT cells induced by TNF-α and IFN-7;

FIG. 28 shows an influence of the exopolysaccharide EPS of the Roseomonas mucosa DL-1 on an atopic dermatitis skin damage of the BALB/c mice induced by MC903;

FIG. 29 shows case analysis of the exopolysaccharide EPS of the Roseomonas mucosa DL-1 on the BALB/c mice with atopic dermatitis induced by MC903;

FIG. 30 shows an influence of the exopolysaccharide EPS of the Roseomonas mucosa DL-1 on an ear thickness of the BALB/c mice with atopic dermatitis induced by MC903;

FIG. 31 shows an influence of the exopolysaccharide EPS of the Roseomonas mucosa DL-1 on mast cell infiltration at a skin damage of the BALB/c mice with atopic dermatitis induced by MC903;

FIG. 32 shows statistic analysis of the exopolysaccharide EPS of the Roseomonas mucosa DL-1 on the mast cell infiltration at the skin damage of the BALB/c mice with atopic dermatitis induced by MC903;

FIG. 33 shows an influence of the exopolysaccharide EPS of the Roseomonas mucosa DL-1 on the mRNA expression of an inflammatory factor at the skin damage of the BALB/c mice with atopic dermatitis induced by MC903;

FIG. 34 shows an influence of the exopolysaccharide EPS of the Roseomonas mucosa DL-1 on a filaggrin expression of the BALB/c mice with atopic dermatitis induced by MC903 (blue: DAPI, and red: filaggrin);

FIG. 35 is a result diagram showing an influence of the live bacterial formulation of the skin symbiotic bacterium R. mucosa DL-1 and the exopolysaccharide DL-EPS of the R. mucosa DL-1 on a toxicity of the HaCaT cells;

FIG. 36 is a result diagram showing an influence of the live bacterial formulation of the skin symbiotic bacterium R. mucosa DL-1 on reactive oxygen species induced by 50 mJ/cm² UVB irradiation;

FIG. 37 is a result diagram showing an influence of the exopolysaccharide DL-EPS of the R. mucosa DL-1 on the reactive oxygen species induced by 50 mJ/cm² UVB irradiation;

FIG. 38 is a result diagram showing an influence of the exopolysaccharide DL-EPS of the skin symbiotic bacterium R. mucosa DL-1 on transcription levels of main inflammatory factors IL-6, IL-1β, TNF-α and IFN-β of the HaCaT cells induced by 50 mJ/cm² UVB irradiation (significant markers: * denotes that P<0.05; ** denotes that P<0.01, *** denotes that P<0.001; and **** denotes that P<0.0001);

FIG. 39 is a result diagram showing an influence of the exopolysaccharide DL-EPS of the skin symbiotic bacterium R. mucosa DL-1 on apoptosis of the HaCaT cells induced by 50 mJ/cm² UVB irradiation;

FIG. 40 shows an influence of the live bacterial formulation of the Roseomonas mucosa R. mucosa DL-1 and the exopolysaccharide DL-EPS of the Roseomonas mucosa R. mucosa DL-1 on pathologies of a skin damage visible to the naked eye and a skin damage under the microscope on a back part of C57BL/6 mice induced by 430 mJ/cm² UVB irradiation;

FIG. 41 is a result diagram showing an influence of the live bacterial formulation of the Roseomonas mucosa R. mucosa DL-1 and the exopolysaccharide DL-EPS of the Roseomonas mucosa R. mucosa DL-1 on the mRNA expression of an inflammatory factor at the skin damage on the back part of the C57BL/6 mice induced by 430 mJ/cm² UVB irradiation (significant markers: * denotes that P<0.05; ** denotes that P<0.01, denotes that P<0.001; and **** denotes that P<0.0001);

FIG. 42 is a result diagram showing an influence of the live bacterial formulation of the Roseomonas mucosa R. mucosa DL-1 and the exopolysaccharide DL-EPS of the Roseomonas mucosa R. mucosa DL-1 on apoptosis of epidermal keratinocytes on the back part of the C57BL/6 mice induced by 430 mJ/cm² UVB irradiation (blue, DAPI; red, PI; green, Pan Cytokeratin);

FIG. 43 shows an effect of the Roseomonas mucosa DL-1 and the exopolysaccharide thereof on expression of a skin barrier repair-related factor involucrin gene IVL and an OVO-like transcription factor 1 gene OVOL1, * denotes that P<0.05, ** denotes that P<0.01, *** denotes that P<0.001, and **** denotes that P<0.0001;

FIG. 44 shows an effect of the Roseomonas mucosa DL-1 and the exopolysaccharide thereof on expression of a skin moisturization-related aquaporin 3 gene AQP3, * denotes that P<0.05, ** denotes that P<0.01, *** denotes that P<0.001, and **** denotes that P<0.0001;

FIG. 45 shows an effect of the Roseomonas mucosa DL-1 and the exopolysaccharide thereof on proliferation of the HaCaT cells, * denotes that P<0.05, ** denotes that P<0.01, *** denotes that P<0.001, and **** denotes that P<0.0001;

FIG. 46 shows an effect of the Roseomonas mucosa DL-1 and the exopolysaccharide thereof on expression of an extracellular matrix-related serine palmitoyltransferase gene SPTSSA, a Mohawk protein gene MKX and a signal transduction protein gene SMAD3 by an H₂O₂-induced HFF cell oxidation model, * denotes that P<0.05, ** denotes that P<0.01, *** denotes that P<0.001, and **** denotes that P<0.0001;

FIG. 47 shows an effect of the Roseomonas mucosa DL-1 and the exopolysaccharide thereof on expression of an antioxidation-related nuclear factor E2-related factor 2 gene NRF2 and an apoptosis inhibition-related B lymphoblastoma-2 gene BCL-2 by the H₂O₂-induced HFF cell oxidation model, * denotes that P<0.05, ** denotes that P<0.01, *** denotes that P<0.001, and **** denotes that P<0.0001;

FIG. 48 shows an effect of the Roseomonas mucosa DL-1 and the exopolysaccharide thereof on expression of cell growth factor-related fibroblast growth factor genes FGF1 and FGF2 and a hepatocyte growth factor gene HGF by the H₂O₂-induced HFF cell oxidation model, * denotes that P<0.05, ** denotes that P<0.01, *** denotes that P<0.001, and **** denotes that P<0.0001;

FIG. 49 shows an effect of the Roseomonas mucosa DL-1 and the exopolysaccharide thereof on up-regulating expression of antibacterial peptide-related genes of the HaCaT cells, such as psoriasin S100A7, 0-defensin 4 DEFB4 and cathelicidin family anti-bacterial peptide LL-37, * denotes that P<0.05, ** denotes that P<0.01, *** denotes that P<0.001, and **** denotes that P<0.0001; and

FIG. 50 shows an effect of the Roseomonas mucosa DL-1 and the exopolysaccharide thereof on expression of a desmoplakin degradation-related gene kallikrein 5 gene KLK5 and a transmembrane adhesive glycoprotein CD44 by a UVB-induced HaCaT cell photoaging model, * denotes that P<0.05, ** denotes that P<0.01, *** denotes that P<0.001, and **** denotes that P<0.0001.

### DETAILED DESCRIPTION

The present invention is further described in detail hereinafter with reference to the drawings and specific embodiments, and the advantages of the above and/or other aspects of the present invention will be clearer.

### Embodiment 1 Screening and Identification of Roseomonas mucosa DL-1

The Roseomonas mucosa DL-1 was a skin symbiotic bacterium screened by the inventor from skin of healthy people by a screening process as follows.

Human epidermis was wiped with a sterile cotton swab for 50 times, and then the sterile cotton swab was immersed in sterile water, and shaken and mixed evenly. The sample solution was diluted gradiently, and diluted solutions at three concentration gradients of 10⁻⁴, 10⁻⁵ and 10⁻⁶ were evenly coated on a solid medium (the medium contained ingredients of: 0.5 g/L yeast extract powder, 0.5 g/L peptone, 0.5 g/L casein hydrolysate, 0.5 g/L glucose, 0.5 g/L soluble starch, 0.3 g/L dipotassium hydrogen phosphate, 0.024 g/L anhydrous magnesium sulfate, 0.3 g/L sodium pyruvate and 15.0 g/L agar, and had a pH value of 7.2±0.2), and cultured in a constant-temperature incubator at 32° C. for 48 hours. The plate was checked every day in this period to observe colony characteristics, single colonies were obtained by streaking on the solid medium plate, and the operation was repeated for three times to obtain single bacterial strains.

The bacterial strains obtained in the previous step were respectively inoculated into a liquid medium (the medium contained ingredients of: 17.0 g/L tryptone, 3.0 g/L soybean peptone, 5.0 g/L sodium chloride, 2.5 g/L dipotassium hydrogen phosphate and 2.5 g/L glucose, and had a pH value of 7.3±0.2), and shaken on a shaker at 37° C. for fermentation culture, separated bacterial cells were resuspended with normal saline and then used for treatment of a skin damage of lupus erythematosus induced by a combination of UVB and Pristane, and single bacteria with the best effect were screened out and named the Roseomonas mucosa DL-1.

It was identified that the Roseomonas mucosa DL-1 had the following properties.

### (1) Morphological Characteristics of Bacterial Colony

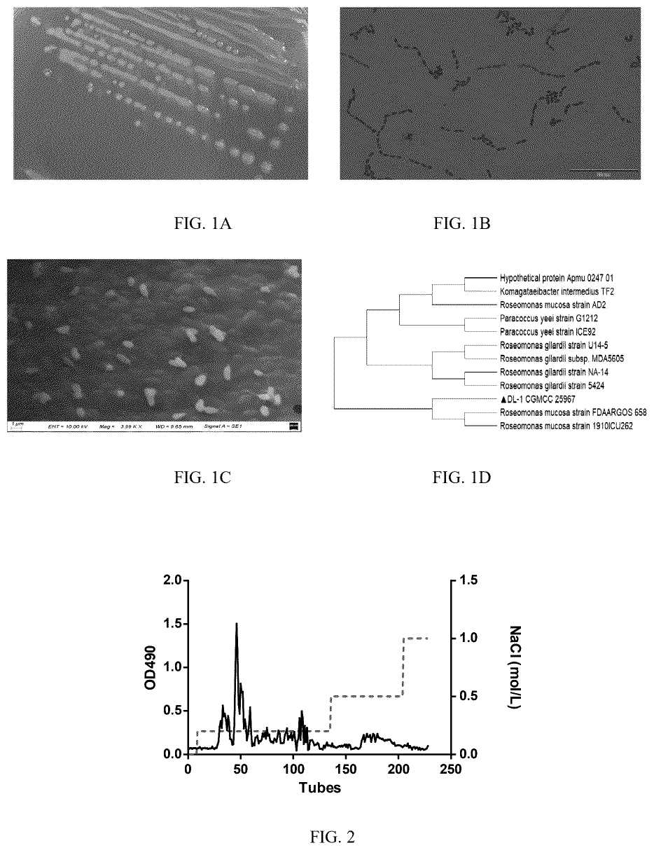

After being cultured in an R2A medium at 32° C. for 16-24 hours, it was observed through a microscope that the bacterial colony was a single bacterial colony, with a morphology as shown in FIG. 1A. Through Gram staining, according to the results as showed in FIG. 1B, the Roseomonas mucosa DL-1 was coccobacillus and belonged to a Gram-negative bacterium. Through scanning electron microscopy, according to the morphology as showed in FIG. 1C, it could be seen that a large number of biological membranes were secreted, the Roseomonas mucosa DL-1 had flagella, thereby being able to move. After being cultured in the above medium at 32° C. for 12 hours, the bacterial cells could grow in large quantities, produced red pigment, and were mucinous in shape and arranged in single, paired or short-chain shaped. A temperature range of growth was 5-40° C., optimally 30-35° C., and a pH range of growth was 5.0-9.5, optimally 6.5-7.5. The bacteria cells could grow normally in LB, R2A, TSB, BAB and other media.

(2) Analysis of 16S rDNA Sequence

A length of the 16S rDNA sequence was 1464 bp. Comparing the 16S rDNA sequence with related species in a GeneBank database, a phylogenetic tree was constructed on the basis of the 16S rDNA sequence, as shown in FIG. 1D. Results showed that a similarity between the bacterial strain DL-1 and the Roseomonas mucosa reached 99.86%. Therefore, the bacterial strain of the present invention was identified as the Roseomonas mucosa, which was classified to Acetobacteraceae in Rhodospirillales, with a strain number of DL-1, and was preserved in China General Microbiological Culture Collection Center, with a preservation date of Oct. 26, 2022 and a preservation number of CGMCC 25967.

### Embodiment 2 Culture of Roseomonas mucosa DL-1

The Roseomonas mucosa DL-1 preserved on a slant at 4° C. was activated, and inoculated on a seed medium (which was an activation medium), wherein an activation temperature was 32° C., and an activation time was 16 hours. A seed fermentation broth was inoculated into a fermentation medium according to a volume ratio of 10%, and cultured at 32° C. for 24 hours under a rotation speed of 200 rpm to obtain a fermentation broth, and a count of live Roseomonas mucosa DL-1 in the fermentation broth reached more than 5×10⁹ CFU/mL.

The activation medium was an R2A medium, and the medium contained ingredients of: 0.5 g/L yeast extract powder, 0.5 g/L peptone, 0.5 g/L casein hydrolysate, 0.5 g/L glucose, 0.5 g/L soluble starch, 0.3 g/L dipotassium hydrogen phosphate, 0.024 g/L anhydrous magnesium sulfate, 0.3 g/L sodium pyruvate and 15.0 g/L agar, and had a pH value of 7.2.

The fermentation medium contained ingredients of 17.0 g/L tryptone, 3.0 g/L soybean peptone, 5.0 g/L sodium chloride, 2.5 g/L dipotassium hydrogen phosphate and 2.5 g/L glucose, and had a pH value of 7.3.

### Embodiment 3 Live Bacterial Formulation of Roseomonas mucosa DL-1

After the fermentation broth obtained in Embodiment 2 was centrifuged (at 9,000 rpm for 5 minutes), the bacterial cells were resuspended in 60% glycerol (by volume fraction) to obtain the, and a count of live Roseomonas mucosa DL-1 reached 1×10⁴-1×10¹¹ CFU/mL.

### Embodiment 4 Preparation of Exopolysaccharide of Roseomonas mucosa DL-1

The fermentation broth in Embodiment 2 was centrifuged (at 3,000-4,000 rpm for 10 minutes) for sterilization. 1-fold volume of the fermentation broth was added with 2-fold volume of sevag reagent (chloroform: n-butanol=4:1, volume ratio), and the operation was repeated for 2-3 times for deproteinization. Then, the fermentation broth was added with 1-fold volume of anhydrous ethanol to culture at 4° C. overnight and centrifuged (at 3,000-4,000 rpm for 10 minutes) to obtain a crude exopolysaccharide precipitate, and the crude exopolysaccharide precipitate was dried, and redissolved with deionized water. About 5-6 g of the crude exopolysaccharide was determined by a sulfuric acid-phenol method, and separated and purified with DEAE centrifugal exchange resin, and an elution curve was as shown in FIG. 2. 0.2 M NaCl elution component was collected and freeze-dried, and then a monosaccharide composition and a molecular weight of the exopolysaccharide EPS were determined respectively.

A monosaccharide determination method for the exopolysaccharide EPS was as follows: a Thermo ICS5000 ion chromatography system (ICS5000, Thermo Fisher Scientific, USA) was used, and monosaccharide components of a mixed standard and a sample were analyzed and detected by an electrochemical detector. Standard substances required for this project were accurately weighed respectively, and then added with water to prepare 10 mg/mL standard substance mother solution single standards, and then, proper amounts of the standard substance mother solution single standards were mixed to prepare a standard substance mixed standard with a highest index concentration of 60 μg/mL, 50 μg/mL or 40 μg/mL. A series of standard substances required for operation on instruments were prepared according to the following concentration gradients.

TABLE 1
Gradient concentration information
of monosaccharide mixed standard

| Name | Abbreviation | Concentration (μg/ml) | | | | | | | |
|---|---|---|---|---|---|---|---|---|---|
| Fucose | Fuc | 0.4 | 0.8 | 4 | 8 | 16 | 24 | 32 | 40 |
| Rhamnose | Rha | 0.4 | 0.8 | 4 | 8 | 16 | 24 | 32 | 40 |
| Arabinose | Ara | 0.4 | 0.8 | 4 | 8 | 16 | 24 | 32 | 40 |
| Galactose | Gal | 0.4 | 0.8 | 4 | 8 | 16 | 24 | 32 | 40 |
| Glucose | Glc | 0.4 | 0.8 | 4 | 8 | 16 | 24 | 32 | 40 |
| Xylose | Xyl | 0.4 | 0.8 | 4 | 8 | 16 | 24 | 32 | 40 |
| Mannose | Man | 0.5 | 1 | 5 | 10 | 20 | 30 | 40 | 50 |
| Fructose | Fru | 0.6 | 1.2 | 6 | 12 | 24 | 36 | 48 | 60 |
| Ribose | Rib | 0.6 | 1.2 | 6 | 12 | 24 | 36 | 48 | 60 |
| Galacturonic acid | Gal-UA | 0.6 | 1.2 | 6 | 12 | 24 | 36 | 48 | 60 |
| Guluronic acid | Gul-UA | 0.6 | 1.2 | 6 | 12 | 24 | 36 | 48 | 60 |
| Glucuronic acid | Glc-UA | 0.6 | 1.2 | 6 | 12 | 24 | 36 | 48 | 60 |
| Mannuronic acid | Man-UA | 0.6 | 1.2 | 6 | 12 | 24 | 36 | 48 | 60 |

The standard substances were mainly from sigma Company.

A Dionex™ CarboPac™ PA20 (150*3.0 mm, 10 m) liquid chromatography column was used; and an injection volume was 5 μl. Mobile phase A (H₂O), mobile phase B (0.1 M NaOH) and mobile phase C (0.1 M NaOH, 0.2 M NaAc), and flow rate: 0.5 ml/min; column temperature: 30° C.; and elution gradients: 0 min A phase/B phase/C phase (95:5:0, V/V), 26 min A phase/B phase/C phase (85:5:10, V/V), 42 min A phase/B phase/C phase (85:5:10, V/V), 42.1 min A phase/B phase/C phase (60:0:40, V/V), 52 min A phase/B phase/C phase (60:40:0, V/V), 52.1 min A phase/B phase/C phase (95:5:0, V/V), and 60 min A phase/B phase/C phase (95:5:0, V/V).

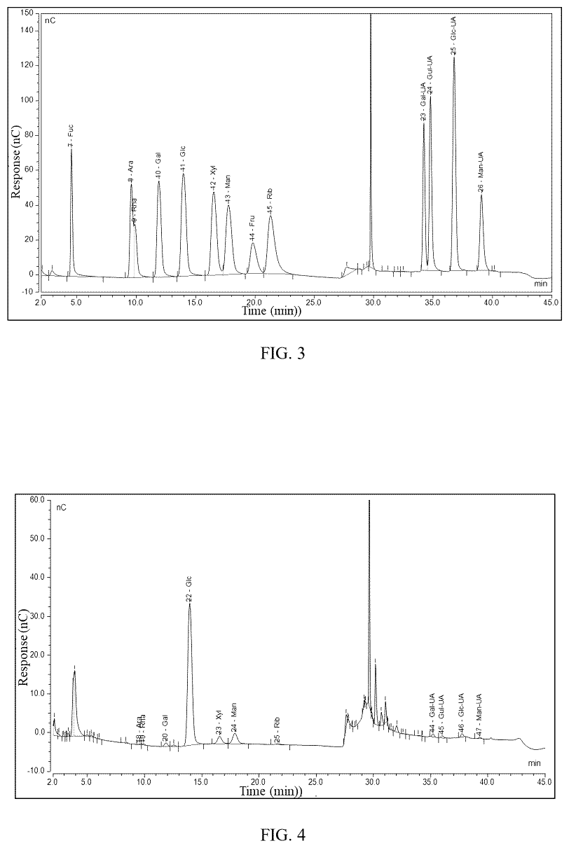

Determination results of the standard substances were as shown in FIG. 3, and 13 standard substances all had a single peak. A chromatogram of the sample was as shown in FIG. 4, which was calculated according to a standard curve of a retention time of the standard substance and a concentration of the sample. It was determined that the monosaccharide composition of the exopolysaccharide EPS was:arabinose:rhamnose:galactose:lucose:xylose:mannose:ribose:alacturonic acid:lucuronic acid:mannuronic acid:uluronic acid=0.51:1.47:1.8:73.98:4.67:9.96:0.94:2.33:1.35:1.32:1.67.

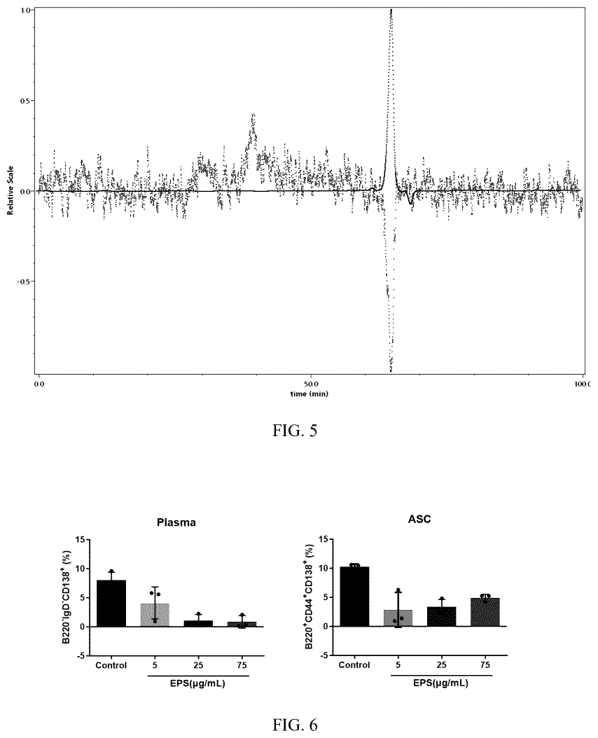

A molecular weight determination method for the exopolysaccharide EPS was s as follows: a gel chromatography-differential-multi-angle laser light scattering system was used as a chromatographic system, a liquid phase system was U3000 (Thermo, USA), a differential detector was Optilab T-rEX (Wyatt technology, CA, USA), and a laser light scattering detector was Dawn Hele OS II (Wyatt Technology, CA, USA). Gel exclusion chromatography columns (Ohpak SB-805 HQ (300×8 mm), Ohpak SB-804 HQ (300×8 mm) and Ohpak SB-803 HQ (300×8 mm) were connected in series. A column temperature was 45° C., an injection volume was 100 μL, there was a mobile phase A (0.02% NaN₃, 0.1 M NaNO₃), a flow rate was 0.4 mL/min, and elution gradients were isocratic within 100 minutes. Results were as shown in FIG. 5. A retention time of the exopolysaccharide EPS sample was 63.5 minutes, it was calculated according to a Mark-Howinkle equation that a weight-average molecular weight of the exopolysaccharide EPS was 3,000 Da, and it was detected that the exopolysaccharide EPS had a single peak, so that it was determined that the exopolysaccharide EPS sample was a pure exopolysaccharide EPS product.

### Embodiment 5 Influence of Exopolysaccharide EPS of Roseomonas mucosa DL-1 on Differentiation of B Cells Induced by IL-4 and R848 In Vitro

In-vitro induced differentiation of B cells: fresh spleens of 3 C57BL/J mice aged 8 weeks were ground and filtered, and then B cells were selected according to a B-cell negative selection kit (Biolegend, 480088); 500 μL of 1640 medium (containing a double antibody, 50 μM β-mercaptoethanol, 30 ng/mL IL-4 and 100 ng/mL R848) was spread in a 24-well plate, and each well was added with 10¹ selected B cells, and synchronously added with 5-75 μg/mL exopolysaccharide EPS of the Roseomonas mucosa DL-1 for treatment, wherein there were 3 biological replicates in each treatment, and no exopolysaccharide was added for the blank group; and the cells were cultured in a cell incubator for 48 hours, and then subjected to flow cytometry.

Results of the flow cytometry were analyzed by Flowjo, and results were as shown in FIG. 6. Compared with the control group, the treatment with 5-75 μg/mL exopolysaccharide EPS of the Roseomonas mucosa DL-1 could significantly down-regulate proportions of plasma cells and antibody-secreting cells, wherein the treatment with 75 μg/mL exopolysaccharide EPS of the Roseomonas mucosa DL-1 could most significantly down-regulate the proportion of plasma cells, and the treatment with 5 g/mL exopolysaccharide EPS of the Roseomonas mucosa DL-1 could most significantly down-regulate the proportion of antibody-secreting cells. Therefore, the exopolysaccharide EPS of the Roseomonas mucosa DL-1 could inhibit the differentiation of the B cells into the plasma cells and the antibody-secreting cells induced in vitro, thereby being possible to have application prospects in autoimmune diseases in which the B cells were over-activated and differentiated.

### Embodiment 6 Influence of Exopolysaccharide EPS of Roseomonas mucosa DL-1 on Activation of T Cells Induced by Anti-CD3 and Anti-CD28 In Vitro

In-vitro activation of T cells: 300 μL of 1×PBS containing 5 μg/mL anti-CD3 was spread in a 24-well plate at 4° C. for 6-12 hours, fresh spleens of 3 C57BL/J mice aged 8 weeks were ground and filtered, and then T cells were selected according to a T-cell negative selection kit (Biolegend, 480033). 1×PBS in each well was removed, 500 μL of 1640 medium (containing a double antibody, 50 μM β-mercaptoethanol and 2 μg/mL anti-CD28) was spread in the 24-well plate, and each well was added with 10⁵ selected T cells, and synchronously added with 5-500 μg/mL exopolysaccharide EPS of the Roseomonas mucosa DL-1 for treatment, wherein there were 3 biological replicates in each treatment, and no exopolysaccharide was added for the blank group. The cells were cultured in a cell incubator for 58 hours, and then subjected to flow cytometry.

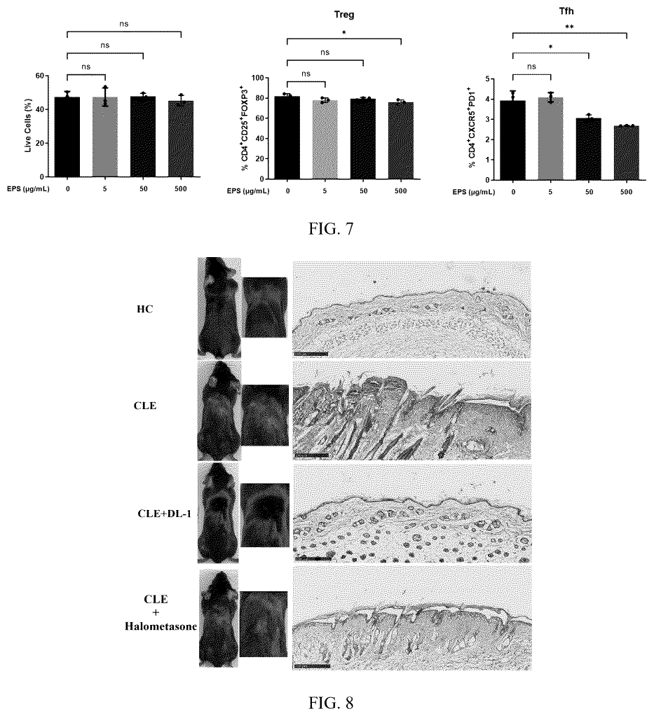

Results of the flow cytometry were analyzed by Flowjo, and results were as shown in FIG. 7. Compared with the control group, the treatment with 5-500 μg/mL exopolysaccharide EPS of the Roseomonas mucosa DL-1 had no influence on cell proliferation and apoptosis during activation of the T cells and did not significantly affect a proportion of Treg cells, but could significantly down-regulate a proportion of Tfh cells, wherein the treatment with 50 μg/mL exopolysaccharide EPS of the Roseomonas mucosa DL-1 could most significantly affect the proportion of Tfh cells during activation of the T cells, thereby being able to inhibit the activation of the T cells into the Tfh cells. Therefore, the exopolysaccharide EPS of the Roseomonas mucosa DL-1 could inhibit the activation of the T cells into the Tfh cells, thereby being possible to have application prospects in autoimmune diseases in which the T cells were over-activated and differentiated.

### Embodiment 7 Capability of Live Bacterial Formulation of Roseomonas mucosa DL-1 to Relieve Cutaneous Lupus Erythematosus Skin Damage

A preparation method for the live bacterial formulation used in the present invention was the same as that in Embodiment 3.

A modeling method for the cutaneous lupus erythematosus referred to the authorized patent (cutaneous lupus erythematosus mouse model, and construction method therefor and application thereof, ZL202110542671.0), and a specific modeling method was as follows.

Feeding environment: SPF grade, mice: C57BL/6 female mice, 8 weeks old; sterile Pristane (Sigma-Aldrich, P2870); and modeling method: (1) 8-week-old C57BL/6 female mice in a uniform size purchased were fed stably for one week, and then back parts of the mice were shaved by 2 cmx 2 cm; (2) each mouse was intradermally injected with Pristane through an insulin needle, with a total of 20-30 injections and a total amount of 250 μL, and immunized for one week; (3) hairless mice were selected to be induced by UVB for 7 days according to an amount of 200 mJ/cm² and an intensity of 900 W/cm²; and (4) hairless mice were selected to be kept under UVB for 14 days according to an amount of 100 mJ/cm² and an intensity of 900 W/cm². 5 treatment methods were set in the experiment, and 6 replicates and 48 mice were set for each treatment method. A UVB irradiance meter used in the above modeling had a model of SIGMA SS-07B (280 nm-320 nm), and was calibrated with a SOLAR irradiance meter in November 2022. 4 treatment methods were set in the experiment, and the administration treatment was unified as follows: the administration was started on the day of UVB irradiation and carried out locally once a day. The treatment methods were as follows:
treatment 1 (HC): no treatment, that was, a healthy control group;
treatment 2 (CLE): Pristane+UVB combined modeling, that was, a CLE model group;
treatment 3 (CLE+Halometasone): Pristane+UVB combined modeling, local application with 50 mg/4 cm² halometasone since UVB irradiation, that was, a positive control group; and
treatment 4 (CLE+DL-1): Pristane+UVB combined modeling, local application with the live bacterial formulation of the Roseomonas mucosa DL-1 (4×10⁹ CFU/mL, 250 μL/4 cm²) since UVB irradiation, that was, a viable bacteria treatment group.

The 4 treatments were consistent in other daily managements. After the experiment was ended, the mice were sacrificed to collect tissue samples, and skin damages of the mice and pathologies of the skin damages were subjected to HE staining analysis. As shown in FIG. 8, compared with the healthy group, the intradermal injection with Pristane for 1 week combined with UVB irradiation could induce a mild CLE skin damage, which was characterized by a dark red scar on a back part of the mice, liquefaction degeneration of basal cells in HE staining, infiltration of a small number of lymphocytes, follicular keratotic plugging, and other typical skin damages. Compared with the CLE model mice, the live bacterial formulation of the Roseomonas mucosa DL-1 improved pathological features, such as bright red or dark red scar-like erythema, follicular keratotic plugging and hyperkeratosis, and had a considerable curative effect compared with the positive control group. It was indicated that the live bacterial formulation of the Roseomonas mucosa DL-1 could relieve the skin damage of the mice with CLE.

### Embodiment 8 Influence of Bacterial Formulation of Roseomonas mucilaginosa DL-1 on Deposition of Immune Complex in Mice with CLE

A back tissue of the mice in different treatment groups in Embodiment 7 was taken for immunofluorescent staining, and an immunofluorescence IgG staining method for the skin tissue of the mice was as follows:
1) a collected back skin damage tissue of the mice in Embodiment 5 was taken out of −80° C. refrigerator, embedded with OCT, and cut into a 5 mm slice by a freezing microtome;
2) the slide was placed in a wet box and rewarmed for 10 minutes;
3) the tissue slice was put into PBS and placed in a horizontal shaker to shake at 80 rpm for 5 minutes each time, in a total of 3 times;
4) after spreading, the PBS around the tissue was wiped off with a paper towel, and a circle was drawn around the tissue with a Pap Pen;
5) the tissue was dropwise added with a QuickBlock™ blocking buffer for Immunol staining, and incubated at room temperature for 10 minutes;
6) the tissue was washed with PBS twice for 5 minutes each time;
7) a directly labeled secondary antibody FITC Goat Anti-Mouse IgG (H+L) (diluted at 1:200, abclonal, article No. AS001) was dropwise added on a surface of the tissue, and the tissue was incubated in a wet box at 37° C. for 60 minutes in the dark;
8) the tissue was washed with PBS for 3 times at 80 rpm for 5 minutes each time; and
9) two drops of DAPI-containing mounting medium were dropwise added on the tissue, the tissue was incubated at room temperature for 5 minutes in the dark to be mounted, and then the mounted tissue was observed under a fluorescent confocal microscope.

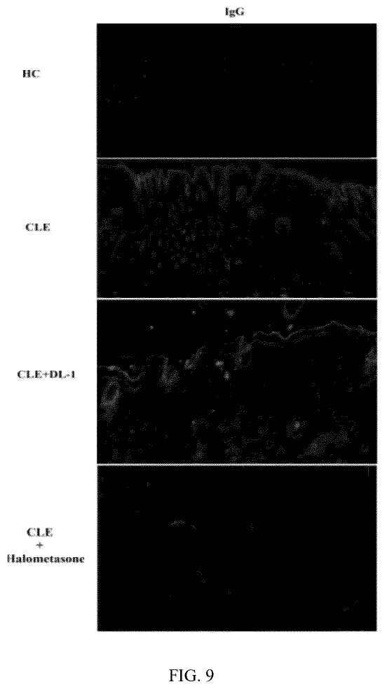

Results were as shown in FIG. 9. Compared with the healthy group, the induction by a combination of Pristane and UVB could lead to deposition of IgG immune complexes in a basement membrane zone at the skin damage on the back part of the mice. Compared with the CLE model mice, the live bacterial formulation of the Roseomonas mucilaginosa DL-1 and the positive control halometasone group both reduced the deposition of the IgG immune complexes in the basement membrane zone at the skin damage on the back part of the mice.

### Embodiment 9 Influence of Roseomonas mucilaginosa DL-1 on Local Immune Cells at Skin Damage of Mice with CLE

Back tissues of 3 mice in each treatment group in Embodiment 7 were taken for a skin flow cytometry experiment, wherein a skin dissociation method was as follows:
(1) the skin tissue of each mouse was washed with 1 mL of HBSS for three times, squeezed to remove droplets as much as possible, transferred to a 6-well plate, and added with 1 mL of precooled skin digestion buffer (containing 100 μg/mL DNase I (ROCHE, 10104159001) and 1 mg/mL Collagenase P (ROCHE, 11213857001), prepared with high-glucose DMEM (Hyclone, SH30022) for current use, and precooled at 4° C.);
(2) the skin tissue was quickly cut into as small pieces as possible on ice by bending scissors;
(3) the skin digestion buffer was added according to an area of the skin damage by 3.5 mL/cm² to incubate the skin tissue at 37° C. for 90 minutes, and the mixture was mixed evenly for many times during digestion;
(4) the mixture was filtered into a 50 mL centrifuge tube by a 40 m cell filter, washed with 12 mL of high-glucose DMEM containing 10% FBS and collected, and centrifuged at 4° C. and 400 g for 5 minutes, the supernatant was discarded, and the cells were resuspended with 400 μL of PBS and counted;

a flow cytometry staining method was as shown in Table 2 below:

TABLE 2

| | | Gating | |
|---|---|---|---|
| marker | Fluorochrome | strategy | |
| CD45 | AF700 | B cell | CD45+CD19+ |
| CD19 | BD | Tfh | CD45+CD4+Bcl6+ICOS+ |
| CD4 | Percp-cy5.5 | Th 1 | CD45+CD4+IFN-r+ |
| IFN-r | PE-CF594 | Th 2 | CD45+CD4+IIL-4+ |
| IL-4 | BV605 | Th 17 | CD45+CD4+IL-17A+ |
| IL-17A | BV421 | | |
| Bcl-6 | APC | | |
| ICOS | PE-cy7 | | |
| LIVE/DIE | APCA750 (live-die | | |
| | cell staining dye) | | |

(5) 200 μL of cells were added into a 24-well plate, added with 300 μL of medium, then added with 0.5 μL of ionomycin Golgi blocker for stimulation (BD, 550583), and incubated at 37° C. for 5-6 hours;
(6) the cell solution was centrifuged at 4° C. and 400 g for 5 minutes, the supernatant was discarded, and the cells were resuspended with 1 mL of 1×PBS, added with 1 μL of Fixable Viability Stain live-die cell staining dye, and incubated at room temperature for 15 minutes in the dark;
(7) the cell solution was centrifuged at 4° C. and 400 g for 5 minutes, the supernatant was discarded, and the cells were added with 0.5 μL of Fc block for vortexing, and incubated at 4° C. for 10 minutes;
(8) flow cytometry antibodies was added: CD45 (9 μL), CD4 (9 μL), ICOS (18 μL) and CD19 (9 μL) were added into 1780 μL of 1×PBS and mixed evenly, and then each sample was added with 100 μL of the mixture, vortexed and incubated at 4° C. for 25 minutes;
(9) the cell solution was added with 1 mL of PBS and centrifuged at 4° C. and 400 g for 5 minutes, the supernatant was discarded, the cells were treated with a nuclear membrane breaking kit (eBioscience, 00-5523-00), and each tube was added with 1 mL of Fixation/Permeabilization working solution (1×) and incubated at 4° C. for 50-60 minutes in the dark;
(10) the cell solution was centrifuged at 400 g for 5 minutes, the supernatant was discarded, and the cells were added with 1 mL of 1× Permeabilization buffer and incubated at 4° C. for 10 minutes;
(11) the cell solution was centrifuged at 500 g for 5 minutes, the supernatant was discarded, the cells were add with IL-4 (18 μL), IL-17A (18 μL), IFN-7 (18 μL) and Bcl-6 (27 μL), and added with 1,760 uL of Perm/Wash Buffer (1×) and mixed evenly, and each sample was added with 100 μL of the mixture, vortexed and incubated at 4° C. for 40-50 minutes; and
(12) the cell solution was centrifuged at 500 g for 5 minutes, the supernatant was discarded, and the cells were added with 1 mL of PBS, filtered by a 70 m cell filter screen, and transferred to a flow cytometry tube, and then the cell solution was centrifuged at 4° C. and 500 g for 5 minutes, the supernatant was discarded, and the cells were added with 200 μL of PBS, vortexed and used for operation on instruments.

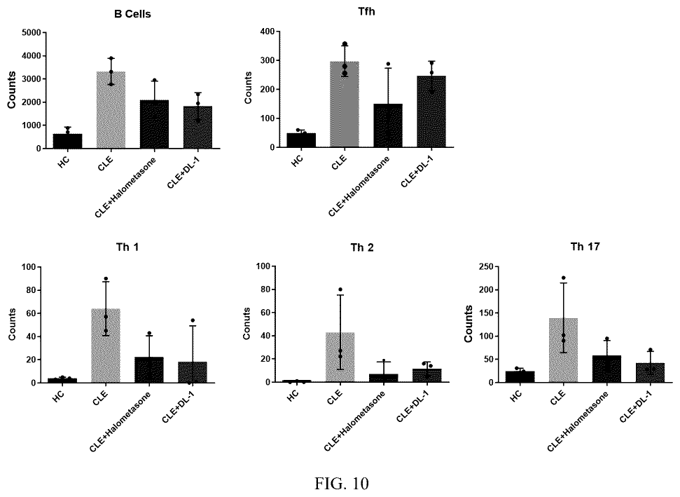

The flow cytometry results were analyzed by Flowjo, and results were as shown in FIG. 10. Compared with the healthy group, the induction by a combination of Pristane and UVB could lead to an increase of a large number of B cells and significant increases of Tfh cells, Th1 cells, Th2 cells and Th17 cells at the skin damage on the back part of the mice. Compared with the CLE model mice, the live bacterial formulation of the Roseomonas mucosa DL-1 and the positive control halometasone treatment could both inhibit increasing trends of local B cells, Tfh cells, Th1 cells, Th2 cells and Th17 cells at the skin damage on the back part of the mice with CLE, thereby having an effect of relieving inflammatory cell infiltration at the CLE skin damage.

### Embodiment 10 Capability of Exopolysaccharide EPS of Roseomonas mucosa DL-1 to Relieve Cutaneous Lupus Erythematosus Skin Damage

A preparation method for the exopolysaccharide EPS of the Roseomonas mucosa DL-1 used in the present invention was the same as that in Embodiment 4.

A modeling method for the cutaneous lupus erythematosus was the same as that in Embodiment 7. However, a UVB radiation intensity meter used in this modeling was a UV-B ultraviolet radiation meter (probe 279) from Beijing Shida Photoelectric Technology Co., Ltd.

4 treatment methods were set in the experiment, and the administration treatment was unified as follows: the administration was started on the day of UVB irradiation, and carried out in a local mode once a day. The treatment methods were as follows:
treatment 1 (HC): no treatment, that was, a healthy control group;
treatment 2 (CLE): Pristane+UVB combined modeling, that was, a CLE model group;
treatment 3 (CLE+Halometasone): Pristane+UVB combined modeling, local application with 50 mg/4 cm² halometasone since UVB irradiation, that was, a positive control group; and
treatment 4 (CLE+EPS): Pristane+UVB combined modeling, local application with the exopolysaccharide EPS of the Roseomonas mucosa DL-1 (40 mg/mL, 25 μL/4 cm²) since UVB irradiation, that was, an exopolysaccharide treatment group.

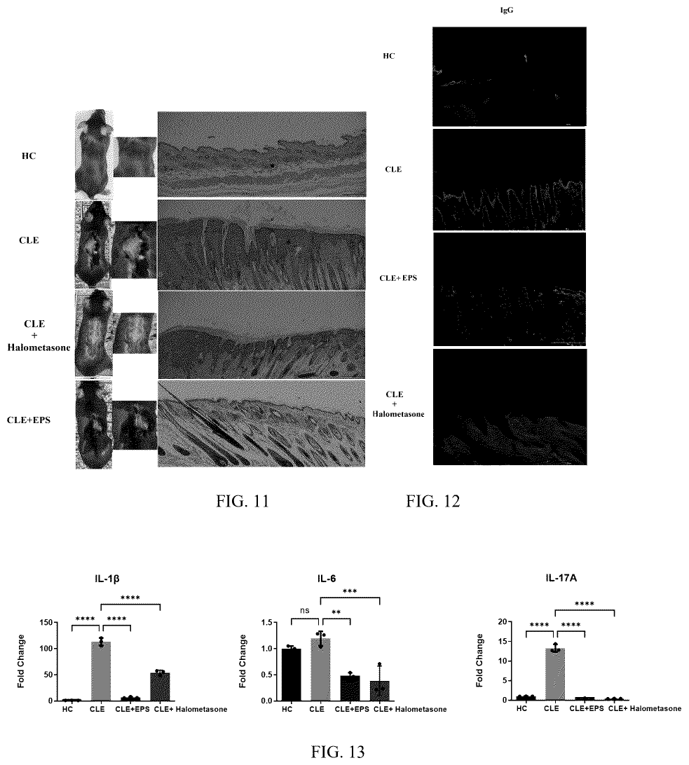

The 4 treatments were consistent in other conventional daily managements. After the experiment was ended, the mice were sacrificed to collect tissue samples, and skin damages of the mice and pathologies of the skin damages were subjected to HE staining analysis. As shown in FIG. 11, compared with the healthy group, the intradermal injection with Pristane for 5 weeks combined with UVB irradiation could induce a severe CLE skin damage, which was characterized by a bright red scar on a back part of the mice, liquefaction degeneration of basal cells in HE staining, infiltration of a large number of lymphocytes, follicular keratotic plugging, and other typical skin damages. Compared with the CLE model mice, the exopolysaccharide EPS of the Roseomonas mucosa DL-1 significantly improved pathological features, such as bright red scar-like erythema, follicular keratotic plugging and hyperkeratosis, and had a significant curative effect compared with the positive control halometasone group, so that the live bacterial formulation of the Roseomonas mucosa DL-1 could relieve the skin damage of the mice with CLE.

### Embodiment 11 Influence of Exopolysaccharide EPS of Roseomonas mucosa DL-1 on Deposition of Immune Complex in Mice with CLE

The skin damage on the back part of the mice in Embodiment 10 was taken, and a staining method for deposition of IgG immune complexes was the same as that in Embodiment 8. Results were as shown in FIG. 12. Compared with the healthy group, the induction by a combination of Pristane and UVB could lead to the deposition of the IgG immune complexes in a basement membrane zone at the skin damage on the back part of the mice. Compared with the matrix treatment group, neither the exopolysaccharide EPS of the Roseomonas mucilaginosa DL-1 nor the positive control halometasone treatment group showed the deposition of the IgG immune complexes in the basement membrane zone at the skin damage on the back part of the mice.

### Embodiment 12 Influence of Exopolysaccharide EPS of Roseomonas mucosa DL-1 on Transcription Levels of Local Inflammatory Factors IL-6, IL-1β and IL-17A in Mice with CLE

A skin damage tissue of the mice in Embodiment 10 was fully ground by a tissue grinder, and then a total RNA of cells was extracted with a TRIZOL kit. The total RNA was reversely transcribed to synthesize a cDNA with a reverse transcription kit, and a target mRNA expression level was detected with an SYBR Green kit on an ABI Prism 7900 real-time quantitative PCR instrument. With β-actin as an internal reference, a relative expression quantity of mRNA of a target gene was calculated according to 2^{-ΔΔCt}.

As shown in FIG. 13, the induction by a combination of Pristane and UVB could significantly increase the transcription levels of the local main inflammatory factors IL-6, IL-1β and IL-17A at the skin damage on the back part of the mice, and the treatment with the exopolysaccharide EPS of the Roseomonas mucosa DL-1 could inhibit transcription increases of the local main inflammatory factors IL-6, IL-1 and IL-17A at the skin damage induced by the combination of Pristane and UVB.

The present invention provided preparation methods for the live bacterial formulation of the skin symbiotic bacterium Roseomonas mucosa and the exopolysaccharide of the Roseomonas mucosa, and applications of the live bacterial formulation of the skin symbiotic bacterium Roseomonas mucosa and the exopolysaccharide of the Roseomonas mucosa in treating the cutaneous lupus erythematosus. Local and topical application with the viable bacterium R. mucosa and the exopolysaccharide thereof to the skin damage of the CLE model mice induced by UVB and Pristane could significantly improve the skin damage of the CLE model mice. The present invention was simple, safe and effective, and provided a new idea for clinical and translational researches of other autoimmune diseases.

### Embodiment 13 Toxicity Assessment of Exopolysaccharide DL-EPS of Roseomonas mucosa DL-1 on HaCaT Cells

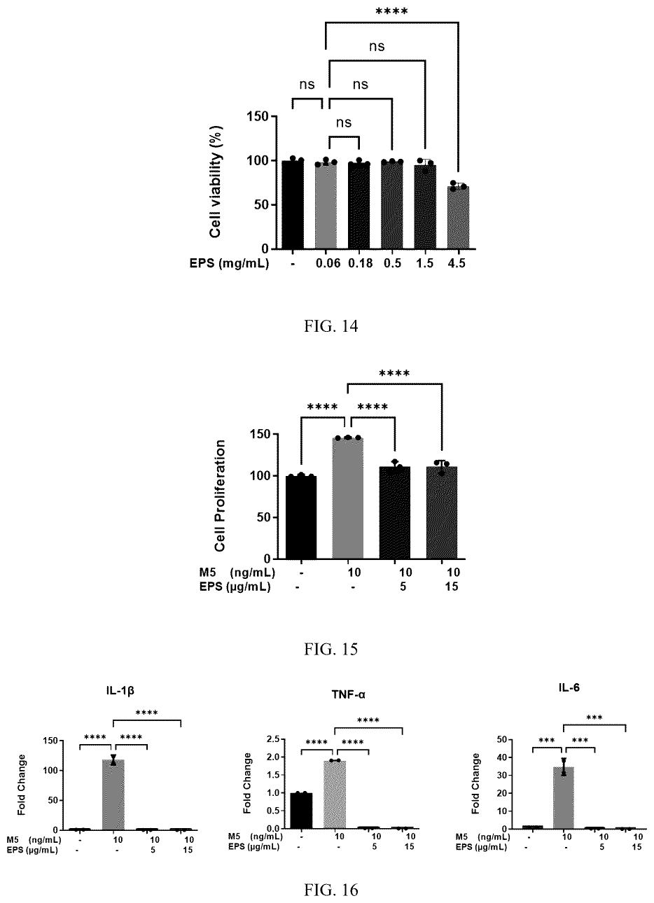

The HaCaT cells were inoculated into a 96-well plate at 1×10⁴ cells/well for 4 hours, and then treated with exopolysaccharides DL-EPS at final concentrations of 0.06-4.5 mg/mL, or added with sterile water as the control, and the cells were incubated in a 5% CO₂ incubator for 24 hours. Each well of the plate was added with 10 μL of CCK8 solution, the culture plate was incubated in an incubator for 1-4 hours, and then an absorbance at 450 nm was measured by a microplate reader (Agilent BioTek, SYNERGY/H1). Results were as shown in FIG. 14. 0.06-1.5 mg/mL exopolysaccharides DL-EPS of the Roseomonas mucosa DL-1 had no significant influence on a viability of the HaCaT cells, which indicated that the exopolysaccharide DL-EPS of the Roseomonas mucosa DL-1 was safe in this concentration range.

The present application further proved that the exopolysaccharide of the Roseomonas mucosa DL-1 inhibited proliferation of the HaCaT cells and the mRNA expression of an inflammatory factor induced by M5, and further disclosed a relieving effect of the exopolysaccharide of the Roseomonas mucosa DL-1 on psoriasis of BALB/c mice induced by imiquimod at the same time.

### Embodiment 14 Influence of Exopolysaccharide DL-EPS of Roseomonas mucosa DL-1 on Proliferation of HaCaT Cells Induced by M5

M5-stimulated 48-hour proliferation experiment: the HaCaT cells were inoculated into a 96-well plate by 5,000 cells/well for 12 hours, and then treated with exopolysaccharides of the Roseomonas mucosa DL-1 at final concentrations of 5 μg/mL and 15 μg/mL, or added with sterile water as the control. 4 hours later, 10 ng/mL M5 was added for stimulation, and 12 hours later, the same amount of exopolysaccharide of the Roseomonas mucosa DL-1 was added for treatment again. The products were respectively cultured in a 5% CO₂ incubator. After stimulation with M5 for 48 hours, the 96-well plate was added with 10 μL of CCK8 solution, the culture plate was incubated in an incubator for 1-4 hours, and then an absorbance at 450 nm was measured by a microplate reader (Agilent BioTek, SYNERGY/H1). The M5 contained TNF-α, oncostain-M, IL-17A, IL-α and IL-22.

Results were as shown in FIG. 15. A viability of the HaCaT cells in the control group was 100%, the M5 could significantly induce the proliferation of the HaCaT cells to 140%, and the exopolysaccharide of the Roseomonas mucosa DL-1 could significantly inhibit the proliferation of the HaCaT cells induced by M5 to recover to near 100% of the control group. The groups both had a significant statistical difference.

### Embodiment 15 Influence of Exopolysaccharide DL-EPS of Roseomonas mucosa DL-1 on Transcription Level of Inflammatory Factor of HaCaT Cells Induced by M5

M5-stimulated 48-hour inflammation experiment: the HaCaT cells were spread in a 6-well culture plate containing 2 mL of 10% FBS-containing DMEM medium at a cell concentration of 1×10⁵ cell/well and cultured overnight, and then four wells were added with the exopolysaccharides DL-EPS of the skin symbiotic bacterium R. mucosa DL-1 obtained in Embodiment 1 at concentrations of 5 μg/mL and 15 μg/mL. 10 ng/mL M5 was added for stimulation, and 12 hours later, the same amount of exopolysaccharide of the Roseomonas mucosa DL-1 was added for treatment again. The products were respectively cultured in a 5% CO₂ incubator. The cells were collected after stimulation with M5 for 48 hours. The M5 contained TNF-α, oncostatin-M, IL-17A, IL-1α and IL-22.

For the cells collected above, a total RNA of the HaCaT cells was extracted with a TRIZOL kit. The total RNA was reversely transcribed to synthesize a cDNA with a reverse transcription kit, and a target mRNA expression level was detected with an SYBR Green kit on an ABI Prism 7900 real-time quantitative PCR instrument. With β-actin as an internal reference, a relative expression quantity of mRNA of a target gene was calculated according to 2^{-ΔΔCt}.

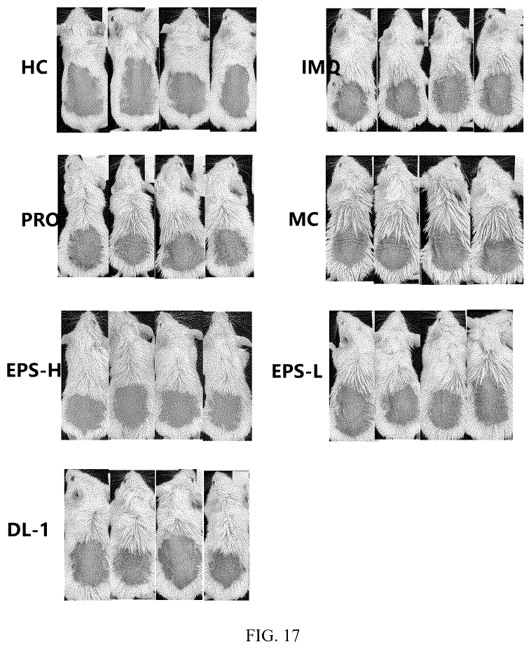

As shown in FIG. 16, transcription levels of inflammatory factors IL-6, IL-1β and TNF-α in the HaCaT cells were induced to increase by more than 40 times, 100 times and 2 times respectively by stimulation with M5 for 48 hours, so that typical characteristics of a psoriasis cell model were induced. Treatments with 5 μg/mL and 15 μg/mL exopolysaccharides DL-EPS of the skin symbiotic bacterium R. mucosa DL-1 could both significantly inhibit the increases of the transcription levels of the inflammatory factors IL-1β, TNF-α and IL-6 in the HaCaT cells induced by stimulation with M5 for 48 hours. The groups both had a statistical significance.

### Embodiment 16 Influences of Roseomonas mucosa DL-1 and Exopolysaccharide DL-EPS of Roseomonas mucosa DL-1 on Psoriasis Skin Damage of BALB/c Mice Induced by Imiquimod

Feeding environment: SPF grade, mice: BALB/c female mice, 8 weeks old; and modeling method: (1) 8-week-old female BALB/c mice in a uniform size purchased were stably fed for one week, then back parts of the mice were shaved by 2 cmx 2 cm, and 62.5 mg of imiquimod was evenly applied to the back part of each mouse every day for 6 consecutive days. 7 treatment methods were set in the experiment, and 4 biological replicates and 28 mice were set for each treatment method. The administration treatment was unified as follows: the administration was carried out locally on the day of modeling, and the administration was carried out every morning and the modeling was carried out every afternoon. The treatment methods were as follows:
treatment 1 (HC): no treatment, that was, a healthy control group;
treatment 2 (IMQ): modeling with imiquimod, that was, a psoriasis model group;
treatment 3 (MC): local and topical administration with 50 mg/4 cm² carbotriol ointment since modeling with imiquimod, that was, a positive control group;
treatment 4 (PRO): local and topical administration with 60% propylene glycol since modeling with imiquimod, that was, a matrix group;
treatment 5 (EPS-H): local and topical administration with 1.5 mg/cm² exopolysaccharide of the Roseomonas mucosa DL-1 since modeling with imiquimod, that was, a high-dose polysaccharide group;
treatment 6 (EPS-L): local and topical administration with 1 mg/cm² exopolysaccharide of the Roseomonas mucosa DL-1 since modeling with imiquimod, that was, a low-dose polysaccharide group; and
treatment 9 (DL-1): local and topical administration with 10⁹ cfu/4 cm² live bacterial formulation of the Roseomonas mucosa DL-1 since modeling with imiquimod, that was, a low-dose polysaccharide group.

The 7 treatments were consistent in other daily managements. After the experiment was ended, the mice were sacrificed to collect tissue samples, and skin damages of the mice and pathologies of the skin damages were subjected to HE staining analysis. As shown in FIG. 17, compared with the healthy group, a typical psoriasis skin damage induced by imiquimod was characterized by epidermal thickness, erythema and scales on the back part of the mice. Compared with the psoriasis model mice, the Roseomonas mucosa DL-1 and the high-dose and low-dose groups of the exopolysaccharide thereof all improved erythema, scales and skin thickening, wherein the high-dose group had a more significant relieving effect. The calcipotriol of the positive control group had a certain curative effect, which was not significant, and long-term treatment with the calcipotriol led to a significant weight loss of the mice, thereby showing a very obvious side effect. It was indicated that the exopolysaccharide of the Roseomonas mucosa DL-1 had a certain relieving effect on a psoriasis-like skin damage of the mice induced by imiquimod, thereby being possible to become a safe and low-toxicity new therapy.

### Embodiment 17 Influences of Roseomonas mucosa DL-1 and Exopolysaccharide DL-EPS Thereof on PASI Scoring of Psoriasis of BALB/c Mice Induced by Imiquimod

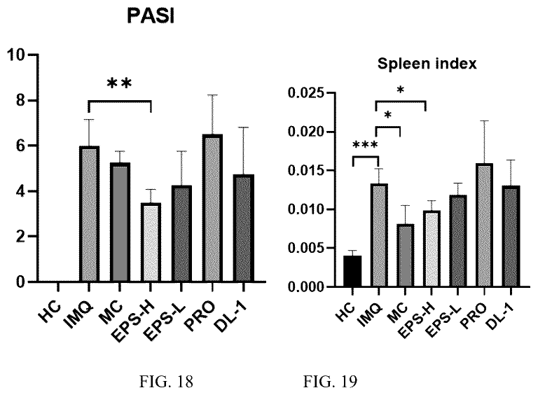

According to the skin damage on the back part of the sacrificed mice in Embodiment 16, erythema, scales and thickening of the skin damage were scored. Statistical results were as shown in FIG. 18. Compared with the healthy group, after continuous application with imiquimod for 6 days, the back part of the mice showed obvious skin damage of erythema, scales and thickening, and the calcipotriol could slightly relieve the erythema, scales and skin thickening of the back part induced by imiquimod. Compared with the matrix group, the exopolysaccharide of the Roseomonas mucosa DL-1 could significantly reduce the score of the skin damage on the back part of the mice, wherein the high-dose treatment group of the exopolysaccharide of the Roseomonas mucosa DL-1 had a statistical difference.

### Embodiment 18 Influences of Roseomonas mucosa DL-1 and Exopolysaccharide DL-EPS of Roseomonas mucosa DL-1 on Spleen Index of BALB/c Mice with Psoriasis Induced by Imiquimod

Statistics was made according to weights and spleen sizes of the sacrificed mice in Embodiment 16. Results were as shown in FIG. 19. Compared with the healthy group, after continuous application with imiquimod for 6 days, the spleen index of the mice was significantly increased, and the calcipotriol could slightly relieve an increase of the spleen index of the mice induced by imiquimod. Compared with the matrix group, the exopolysaccharide of the Roseomonas mucosa DL-1 could significantly reduce the spleen index of the mice, wherein the high-dose treatment group (EPS-H) of the exopolysaccharide of the Roseomonas mucosa DL-1 had a statistical difference.

### Embodiment 19 Influence of Exopolysaccharide DL-EPS of Roseomonas mucosa DL-1 on Pathology Under Microscope at Psoriasis Skin Damage of BALB/c Mice Induced by Imiquimod

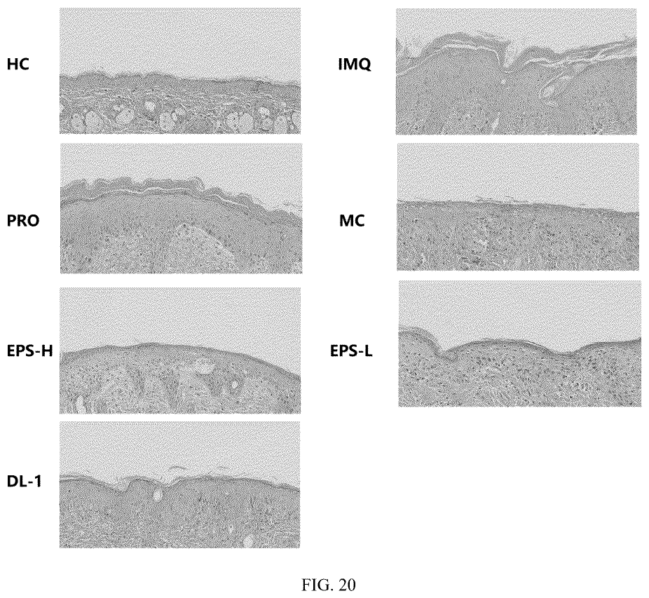

The skin damage of the sacrificed mice in Embodiment 16 was subjected to HE staining. Results of typical pathology were as shown in FIG. 20. Compared with the healthy group, after continuous application with imiquimod for 6 days, the mice showed scale increase and epidermal thickness increase, and the calcipotriol could relieve the scale increase and the epidermal thickness increase of the mice induced by imiquimod. Compared with the matrix group, the Roseomonas mucosa DL-1 and the treatment groups of the exopolysaccharide DL-EPS of the Roseomonas mucosa DL-1 could all reduce a psoriasis-like skin damage induced by imiquimod, wherein the high-dose treatment group (EPS-H) of the exopolysaccharide of the Roseomonas mucosa DL-1 had the most significant curative effect.

### Embodiment 20 Influence of Exopolysaccharide DL-EPS of Roseomonas mucosa DL-1 on Pathology Under Microscope at Psoriasis Skin Damage of BALB/c Mice Induced by Imiquimod

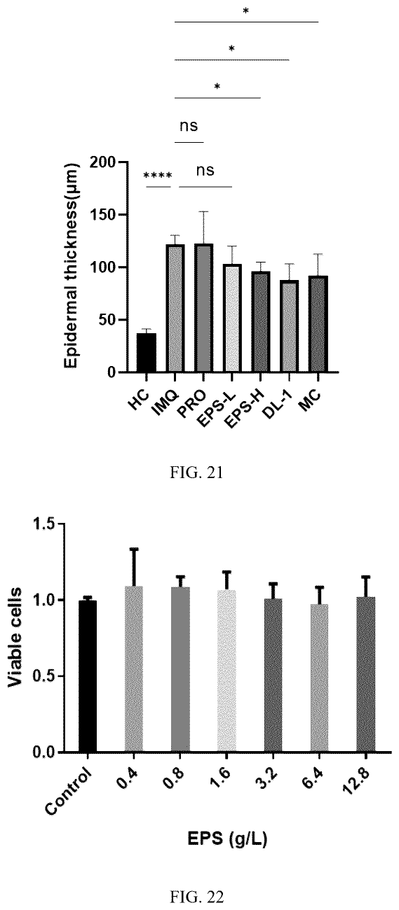

An HE staining image of the skin damage of the sacrificed mice in Embodiment 19 was processed by Image J to statistically analyze an epidermal thickness. Results were as shown in FIG. 21. Compared with the healthy group, after continuous application with imiquimod for 6 days, the mice showed significant epidermal thickening. Compared with the matrix group, excluding the low-dose treatment group (EPS-L) of the exopolysaccharide of the Roseomonas mucosa, the calcipotriol, the Roseomonas mucosa DL-1 and the high-dose treatment group (EPS-H) of the exopolysaccharide DL-EPS could all relieve the epidermal thickening of the mice induced by imiquimod, and had a significant difference.

In a case of being applied as a drug for relieving atopic dermatitis, an inhibiting effect of the exopolysaccharide of the Roseomonas mucosa DL-1 on the mRNA expression of an inflammatory factor of HaCaT cells induced by TNF-α and IFN-7 was disclosed, and a relieving effect of the exopolysaccharide of the Roseomonas mucosa DL-1 on atopic dermatitis of BALB/c mice induced by MC903 was further disclosed.

### Embodiment 21 Toxicity Assessment of Exopolysaccharide of Roseomonas mucosa DL-1 on Raw 264.7 Cells

The Raw 264.7 cells were inoculated into a 96-well plate at 1×10⁴ cells/well for 4 hours, and then treated with exopolysaccharides EPS at final concentrations of 0.4-12.8 μg/mL, or added with sterile water as the control, and the cells were incubated in a 5% CO₂ incubator for 24 hours. Each well of the plate was added with 10 μL of CCK8 solution, the culture plate was incubated in an incubator for 1-4 hours, and then an absorbance at 450 nm was measured by a microplate reader (Agilent BioTek, SYNERGY/H1). Results were as shown in FIG. 22. 0.4-12.8 g/L exopolysaccharides EPS of the Roseomonas mucosa DL-1 had no significant influence on a viability of the Raw 264.7 cells, which indicated that the exopolysaccharide EPS of the Roseomonas mucosa DL-1 was safe in this concentration range.

### Embodiment 22 Influence of Exopolysaccharide of Roseomonas mucosa DL-1 on Transcription Level of Inflammatory Factor of Macrophages Raw 264.7 Induced by Lipopolysaccharide

4-hour lipopolysaccharide (LPS) stimulation: the Raw 264.7 cells were spread in a 6-well culture plate containing 2 mL of 10% FBS-containing DMEM medium at a cell concentration of 1×10⁵ cell/well and cultured overnight, and then four wells were added with the exopolysaccharides EPS of the skin symbiotic bacterium R. mucosa DL-1 obtained in Embodiment 1 at concentrations of 0.75 mg/mL, 1.5 mg/mL, 3 mg/mL and 6 mg/mL. After pretreatment with the exopolysaccharide EPS of the R. mucosa DL-1 for 4 hours, the same well was added with 100 ng/mL lipopolysaccharide (MCE, HY-D1056) for stimulation, and only the lipopolysaccharide was added for stimulation in the model group. The cells were collected after stimulation for 4 hours and 24 hours.

24-hour lipopolysaccharide (LPS) stimulation: the Raw 264.7 cells were spread in a 6-well culture plate containing 2 mL of 10% FBS-containing DMEM medium at a cell concentration of 1×10⁵ cell/well and cultured overnight, and then four wells were added with the exopolysaccharides EPS of the skin symbiotic bacterium R. mucosa DL-1 obtained in Embodiment 1 at concentrations of 0.5 mg/mL and 1.5 mg/mL. After pretreatment with the exopolysaccharide EPS of the R. mucosa DL-1 for 4 hours, the same well was added with 100 ng/mL lipopolysaccharide (MCE, HY-D1056) for stimulation, and only the lipopolysaccharide was added for stimulation in the model group. After stimulation for 4 hours, the cells were treated with the same amount of exopolysaccharide, and the cells were collected after stimulation with the lipopolysaccharide for a total of 24 hours.

For the cells collected above, a total RNA of the Raw 264.7 cells was extracted with a TRIZOL kit. The total RNA was reversely transcribed to synthesize a cDNA with a reverse transcription kit, and a target mRNA expression level was detected with an SYBR Green kit on an ABI Prism 7900 real-time quantitative PCR instrument. With β-actin as an internal reference, a relative expression quantity of mRNA of a target gene was calculated according to 2^{-ΔΔCt}.

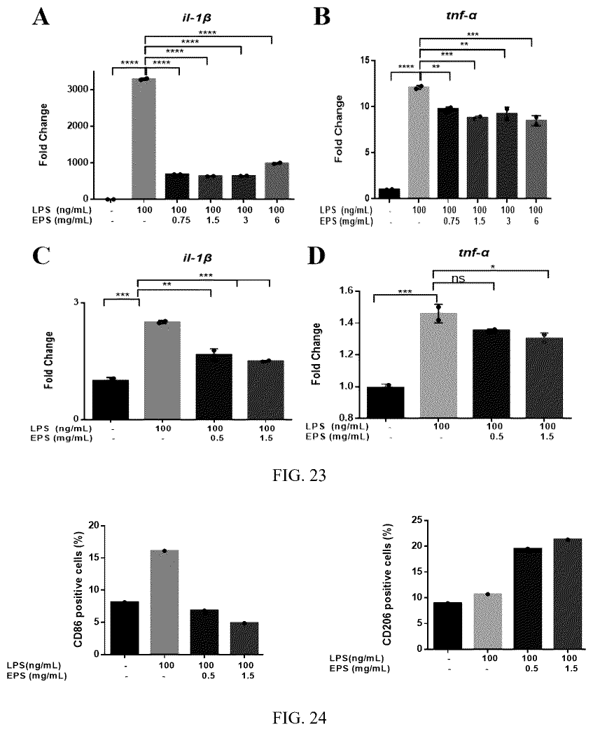

As shown in FIG. 23A-B, transcription levels of inflammatory factors IL-1β and TNF-α in the Raw 264.7 cells were induced to increase by more than 3,000 times and 12 times respectively by stimulation with the lipopolysaccharide for 4 hours, and treatments with 0.75 mg/mL, 1.5 mg/mL, 3 mg/mL and 6 mg/mL exopolysaccharides EPS of the skin symbiotic bacterium R. mucosa DL-1 could all significantly inhibit increases of the transcription levels of the inflammatory factors IL-1β and TNF-α in the Raw 264.7 cells induced by stimulation with the lipopolysaccharide for 4 hours. The groups all had a statistical significance. As shown in FIG. 23C-D, the transcription levels of the inflammatory factors IL-1β and TNF-α in the Raw 264.7 cells were induced to increase by stimulation with the lipopolysaccharide for 24 hours, and treatments with 0.5 mg/mL and 1.5 mg/mL exopolysaccharides EPS of the skin symbiotic bacterium R. mucosa DL-1 could both inhibit increases of the transcription levels of the inflammatory factors IL-1β and TNF-α in the Raw 264.7 cells induced by stimulation with the lipopolysaccharide for 24 hours, wherein the treatment group of the 1.5 mg/mL exopolysaccharide EPS of the skin symbiotic bacterium R. mucosa DL-1 had a statistical significance.

The Raw 264.7 cells stimulated by the lipopolysaccharide (LPS) for 24 hours were collected for flow cytometry staining by a method as follows.

The cells were collected and centrifuged at 4° C. and 400 g for 5 minutes, the supernatant was discarded, and the cells were resuspended with 1 mL of 1×PBS, added with 1 μL of Fixable Viability Stain BV510 live-die cell staining dye, and incubated at room temperature for 15 minutes in the dark.

The cell solution was centrifuged at 4° C. and 400 g for 5 minutes, the supernatant was discarded, and the cells were added with 0.5 μL of Fc block for vortexing, and incubated at 4° C. for 10 minutes.

6 μL of CD 86 (Biolegend, article No. 105032) and 6 μL of CD 206 (Invitrogen, article No. 2175437) were added into 610 μL of 1×PBS and mixed evenly, and then each sample was added with 100 μL of the mixture for vortexing, and incubated at 4° C. for 25 minutes.

The cell solution was centrifuged at 500 g for 5 minutes, the supernatant was discarded, and the cells were added with 1 mL of PBS, filtered by a 70 m cell filter screen, and transferred to a flow cytometry tube, and then the cell solution was centrifuged at 4° C. and 500 g for 5 minutes, the supernatant was discarded, and the cells were added with 200 μL of PBS, vortexed and used for instrument operation.

Flow cytometry analysis was carried out by Flowjo. Results were as shown in FIG. 24. The stimulation with the lipopolysaccharide for 24 hours could significantly induce polarization of the Raw 264.7 cells to M1, and the treatments with the 0.5 mg/mL and 1.5 mg/mL exopolysaccharides EPS of the skin symbiotic bacterium R. mucosa DL-1 could both significantly inhibit polarization of the Raw 264.7 cells to M1 and promote polarization of the Raw 264.7 cells to M2, so that the exopolysaccharide EPS of the skin symbiotic bacterium R. mucosa DL-1 had an application prospect in anti-inflammation.

### Embodiment 23 Influence of Exopolysaccharide of Roseomonas mucosa DL-1 on Transcription Level of Inflammatory Factor of Macrophages Raw 264.7 Induced by Lipopolysaccharide and IFN-γ

24-hour lipopolysaccharide and IFN-7 stimulation: the Raw 264.7 cells were spread in a 6-well culture plate containing 2 mL of 10% FBS-containing DMEM medium at a cell concentration of 1×10¹ cell/well and cultured overnight, and then four wells were added with the exopolysaccharides EPS of the skin symbiotic bacterium R. mucosa DL-1 obtained in Embodiment 1 at concentrations of 0.5 mg/mL and 1.5 mg/mL. After pretreatment with the exopolysaccharide EPS of the R. mucosa DL-1 for 4 hours, the same well was added with 10 ng/mL lipopolysaccharide (MCE, HY-D1056) and 10 ng/mL IFN-7 (ABelonal, RP01070) for stimulation, and only the lipopolysaccharide and IFN-7 were added for stimulation in the model group. After stimulation for 4 hours, the cells were treated with the same amount of exopolysaccharide, and the cells were collected after stimulation with the lipopolysaccharide and IFN-γ for a total of 24 hours. RNA and RT-PCR operations of the cells were the same as those in Embodiment 22.

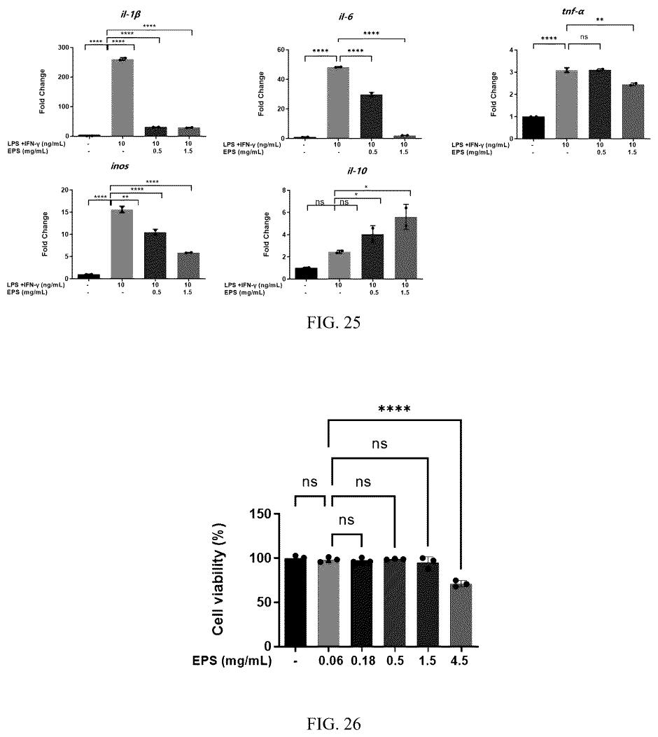

As shown in FIG. 25, the mRNA expression of an inflammatory factor IL-1β in the Raw 264.7 cells was induced to increase by more than 300 times by co-stimulation with 10 ng/mL lipopolysaccharide and 10 ng/mL IFN-γ for 24 hours, and treatments with 0.5 mg/mL and 1.5 mg/mL exopolysaccharides EPS of the skin symbiotic bacterium R. mucosa DL-1 could both inhibit increases of transcription levels of inflammatory factors IL-6, IL-1, INOS and TNF-α in the Raw 264.7 cells induced by the lipopolysaccharide and IFN-7, wherein the treatment group of the 1.5 mg/mL exopolysaccharide EPS of the skin symbiotic bacterium R. mucosa DL-1 had a better effect. The groups both had a statistical significance. The treatment with the 1.5 mg/mL exopolysaccharide EPS of the skin symbiotic bacterium R. mucosa DL-1 could significantly increase the mRNA expression of IL-10 in the Raw 264.7 cells, so that the exopolysaccharide EPS of the skin symbiotic bacterium R. mucosa DL-1 had application prospects in anti-inflammation, anti-infection, and other aspects.

### Embodiment 24 Toxicity Assessment of Exopolysaccharide of Roseomonas mucosa DL-1 on HaCaT Cells

The HaCaT cells were inoculated into a 96-well plate at 1×10⁴ cells/well for 4 hours, and then treated with exopolysaccharides EPS at final concentrations of 0.06-4.5 mg/mL, or added with sterile water as the control, and the cells were incubated in a 5% CO₂ incubator for 24 hours. Each well of the plate was added with 10 μL of CCK8 solution, the culture plate was incubated in an incubator for 1-4 hours, and then an absorbance at 450 nm was measured by a microplate reader (Agilent BioTek, SYNERGY/H1). Results were as shown in FIG. 26. 0.06-1.5 mg/mL exopolysaccharides EPS of the Roseomonas mucosa DL-1 had no significant influence on a viability of the HaCaT cells, which indicated that the exopolysaccharide EPS of the Roseomonas mucosa DL-1 was safe in this concentration range.

### Embodiment 25 Influence of Exopolysaccharide of Roseomonas mucosa DL-1 on Transcription Level of Inflammatory Factor of HaCaT Cells Induced by TNF-α and IFN-7

24-hour TNF-α and IFN-γ stimulation: the HaCaT cells were spread in a 6-well culture plate containing 2 mL of 10% FBS-containing DMEM medium at a cell concentration of 1×10⁵ cell/well and cultured overnight, and then four wells were added with the exopolysaccharides EPS of the skin symbiotic bacterium R. mucosa DL-1 obtained in Embodiment 1 at concentrations of 0.5 μg/mL, 5 μg/mL, 50 μg/mL and 500 μg/mL. After pretreatment with the exopolysaccharide EPS of the R. mucosa DL-1 for 4 hours, the same well was added with 10 ng/mL TNF-α and 10 ng/mL IFN-γ for stimulation, and only TNF-α and IFN-γ were added for stimulation in the model group. The cells were collected after stimulation for 24 hours.

For the cells collected above, a total RNA of the HaCaT cells was extracted with a TRIZOL kit. The total RNA was reversely transcribed to synthesize a cDNA with a reverse transcription kit, and a target mRNA expression level was detected with an SYBR Green kit on an ABI Prism 7900 real-time quantitative PCR instrument. With β-actin as an internal reference, a relative expression quantity of mRNA of a target gene was calculated according to 2^{-ΔΔCt}.

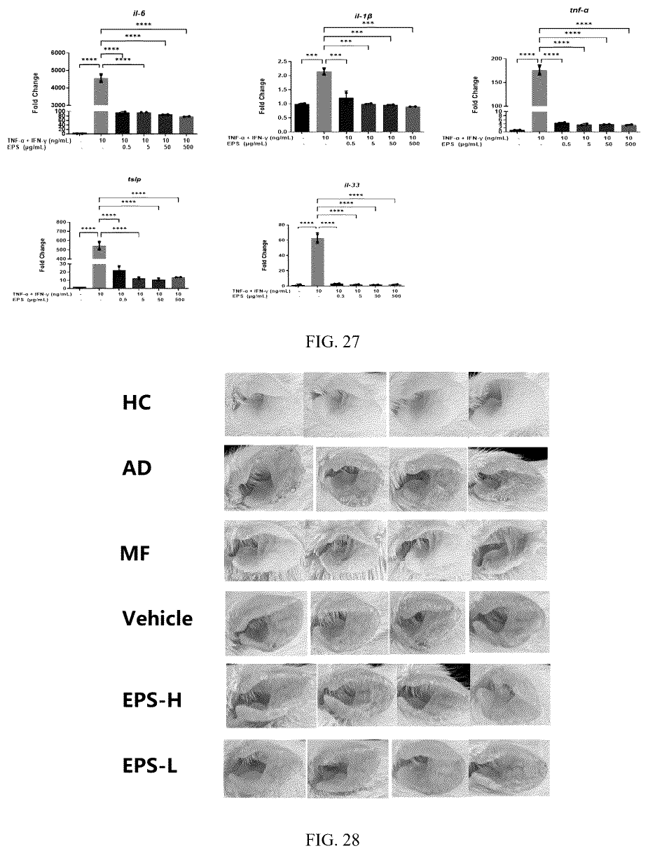

As shown in FIG. 27, transcription levels of inflammatory factors IL-6, IL-1β, TNF-α, TSLP and IL-33 in the HaCaT cells were induced to increase by more than 4,000 times, 2 times, more than 170 times, more than 50 times and more than 60 times respectively by stimulation with TNF-α and IFN-γ for 24 hours, so that typical characteristics of an AD cell model were induced. Treatments with 0.5 μg/mL, 5 μg/mL, 50 μg/mL and 500 g/mL exopolysaccharides of the skin symbiotic bacterium R. mucosa DL-1 could all significantly inhibit increases of the transcription levels of the inflammatory factors IL-13, TNF-α, IL-6, TSLP and IL-33 in the HaCaT cells induced by stimulation with TNF-α and IFN-γ for 24 hours. The groups all had a statistical significance, wherein the treatment groups of the 50 μg/mL and 500 μg/mL exopolysaccharides of the skin symbiotic bacterium R. mucosa DL-1 had a better effect.

### Embodiment 26 Influence of Exopolysaccharide of Roseomonas mucosa DL-1 on Skin Damage of BALB/c Mice with Atopic Dermatitis Induced by MC903

Feeding environment: SPF grade, mice: BALB/c female mice, 8 weeks old; and modeling method: (1) 8-week-old female BALB/c mice in a uniform size purchased were stably fed for one week, and then 1 nmol MC903 was evenly applied to an ear of each mouse every day. 5 treatment methods were set in the experiment, and 4 biological replicates and 24 mice were set for each treatment method. The administration treatment was unified as follows: the administration was carried out locally on the day of modeling, and the administration was carried out every morning and the modeling was carried out every afternoon. The treatment methods were as follows:

treatment 1 (HC): no treatment, that was, a healthy control group;
treatment 2 (AD): modeling with MC903, that was, an AD model group;
treatment 3 (MF): local and topical administration with mometasone furoate since modeling with MC903, that was, a positive control group;
treatment 4 (Vehicle): local and topical administration with 60% propylene glycol since modeling with MC903, that was, a matrix group;
treatment 5 (EPS-H): local and topical administration with 40 mg/cm² exopolysaccharide of the Roseomonas mucosa DL-1 since modeling with MC903, that was, a high-dose polysaccharide group; and
treatment 6 (EPS-L): local and topical administration with 40 mg/cm² exopolysaccharide of the Roseomonas mucosa DL-1 since modeling with MC903, that was, a low-dose polysaccharide group.

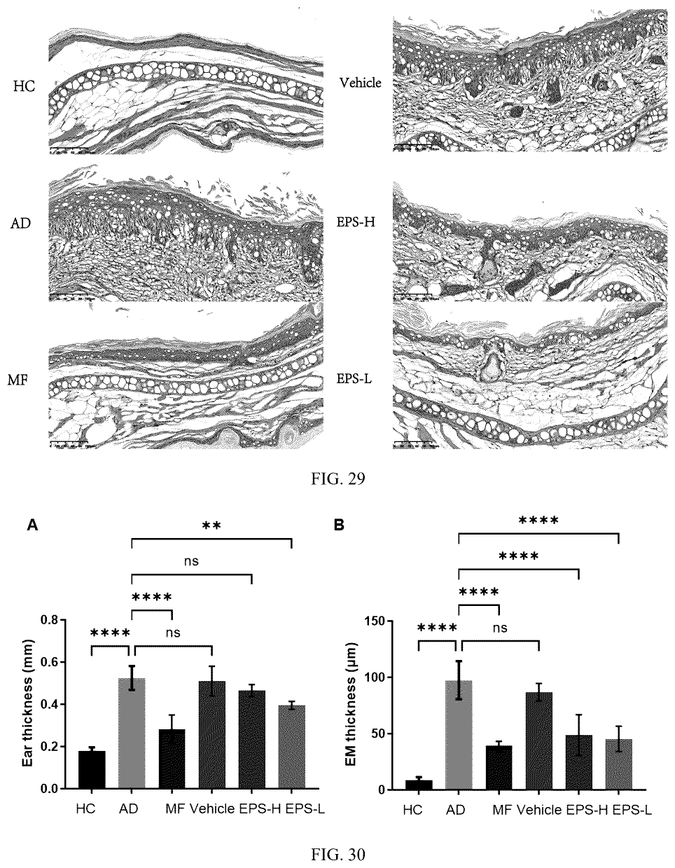

The 6 treatments were consistent in other daily managements. After the experiment was ended, the mice were sacrificed to collect tissue samples, and skin damages of the mice and pathologies of the skin damages were subjected to HE staining analysis. As shown in FIG. 28, compared with the healthy group, a typical AD skin damage induced by MC903 was characterized by ear thickening, erythema and scales of the mice. Compared with the AD model mice, the high-dose and low-dose groups of the exopolysaccharide of the Roseomonas mucosa DL-1 both improved erythema and scales, wherein the low-dose group had a more significant relieving effect. The mometasone furoate of the positive control group had a very significant curative effect, but long-term treatment with the hormone showed a side effect of atrophoderma. Pathological analysis was as shown in FIG. 29. Ear epidermis and dermis swelling of the mice could be induced by MC903, with severe inflammatory cell infiltration. Compared with the matrix treatment group, the high-dose and low-dose EPS-H treatment groups could both improve the ear swelling and the inflammatory cell infiltration of the mice, wherein the low-dose EPS-L treatment group had a more significant effect. Therefore, the exopolysaccharide of the Roseomonas mucosa DL-1 had a certain relieving effect on an AD-like skin damage of the mice induced by MC903, thereby being possible to become a safe and low-toxicity new therapy.

### Embodiment 27 Influence of Exopolysaccharide of Roseomonas mucosa DL-1 on Ear Thickness and Epidermal Thickening of BALB/c Mice with Atopic Dermatitis Induced by MC903

After induction by MC903 for 15 days, the ear thickness of the mice in Embodiment 5 was measured by a vernier caliper, and the each thickness of each mouse was measured for 3 times to get a mean value. Statistical results were as shown in FIG. 30A. Compared with the healthy group, after continuous application with MC903 for 15 days, the mice showed significant ear thickening, and the mometasone furoate could significantly inhibit the ear thickening induced by MC903. Compared with the matrix group, the exopolysaccharide of the Roseomonas mucosa DL-1 could relieve the ear thickening of the mice induced by MC903, wherein the low-dose treatment group of the exopolysaccharide of the Roseomonas mucosa DL-1 had a statistical difference. A pathological image in Embodiment 26 was processed by Image J to statistically analyze an epidermal thickness. Results were as shown in FIG. 30B. Compared with the healthy group, after continuous application with MC903 for 15 days, the mice showed significant epidermal thickening. Compared with the matrix group, the mometasone furoate and the exopolysaccharide EPS treatment groups could all relieve the epidermal thickening of the mice induced by MC903. The groups all had a significant difference, wherein an average epidermal thickness in the low-dose exopolysaccharide EPS-L treatment group was lower than that in the high-dose exopolysaccharide EPS-H treatment group.

### Embodiment 28 Influence of Exopolysaccharide of Roseomonas mucosa DL-1 on Mast Cell Infiltration at Skin Damage of BALB/c Mice with Atopic Dermatitis Induced by MC903

The skin damage tissue of the sacrificed mice in Embodiment 26 was embedded in paraffin and then sliced, and subjected to mast cell staining through toluidine blue by a staining method as follows: (1) the tissue was fixed in a neutral formaldehyde solution or formaldehyde ethanol solution; (2) the tissue slice was deparaffinated; (3) the tissue slice was placed into a toluidine blue solution (0.5 g of toluidine blue added with distilled water to a volume of 100 ml) to stain for 30 minutes; (4) the tissue slice was slightly washed with water; (5) the tissue slice was differentiated with a glacial acetic acid solution (0.5 ml of glacial acetic acid added with distilled water to a volume of 100 ml) until cell nuclei and granules were clear (under control of a microscope); (6) the tissue slice was slightly washed with water, and dried with cold air; and (7) the tissue slice was mounted with xylene or transparent neutral gum.

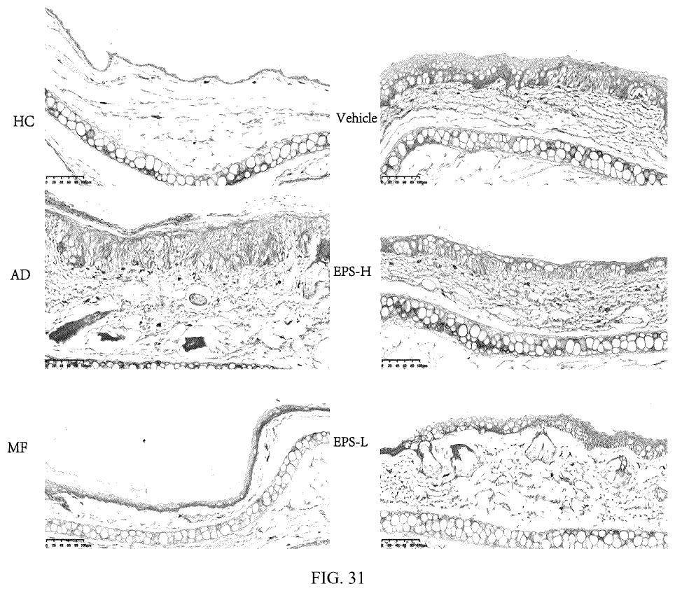

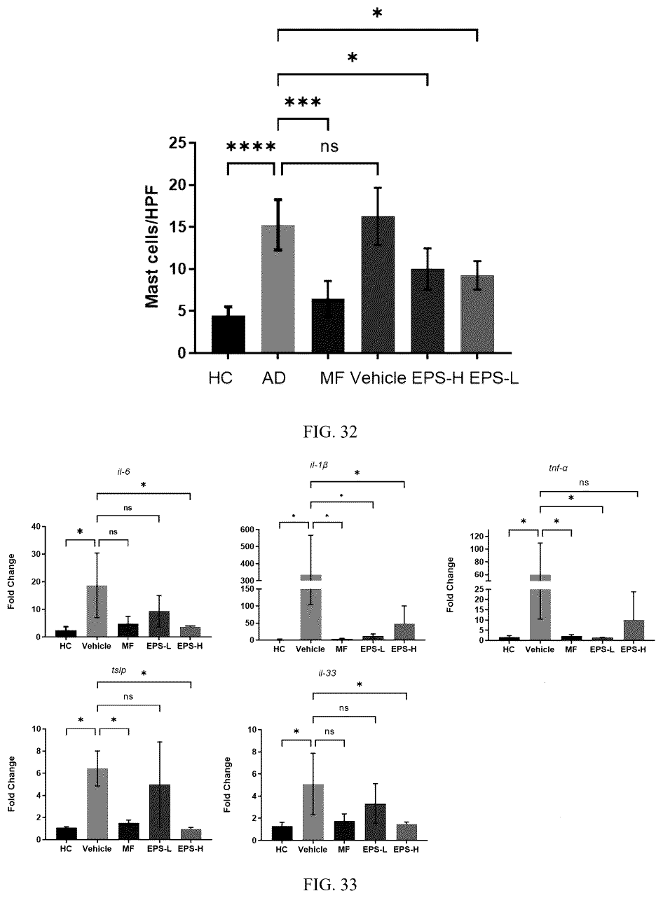

Results of statistical analysis were as shown in FIG. 31. Mast cell granules were reddish violet and mast cell nuclei were blue. Treatment with MC903 could significantly induce mast cell infiltration in dermis of the mice. Compared with the matrix treatment group, the mometasone furoate and the exopolysaccharide EPS treatment groups could all relieve the mast cell infiltration of the mice induced by MC903. A mean value of mast cell counting under each high-power microscope after full slice scanning was calculated. Statistical results were as shown in FIG. 32. The mometasone furoate and the exopolysaccharide EPS treatment groups all had a significant difference, wherein an effect of the low-dose exopolysaccharide EPS-L treatment group was better than that of the high-dose exopolysaccharide EPS-H treatment group.

### Embodiment 29 Influence of Exopolysaccharide of Roseomonas mucosa DL-1 on Transcription Level of Inflammatory Factor at Skin Damage of BALB/c Mice with Atopic Dermatitis Induced by MC903

A skin damage tissue of the sacrificed mice in the five groups (the healthy group, the matrix treatment group, the mometasone furoate treatment group and the exopolysaccharide treatment groups) in Embodiment 26 was ground, and a total RNA of the skin damage tissue was extracted with a TRIZOL kit. The total RNA was reversely transcribed to synthesize a cDNA with a reverse transcription kit, and a target mRNA expression level was detected with an SYBR Green kit on an ABI Prism 7900 real-time quantitative PCR instrument. With β-actin as an internal reference, a relative expression quantity of mRNA of a target gene was calculated according to 2^{-ΔΔCt}.

Results were as shown in FIG. 33. Transcription levels of inflammatory factors IL-6, IL-13, TNF-α, TSLP and IL-33 in an ear part of the BALB/c mice could be induced to increase significantly by MC903. Compared with the matrix treatment group, the EPS treatment groups and the mometasone furoate treatment group could all inhibit increases of the transcription levels of the inflammatory factors induced by MC903. According to statistical analysis, the positive mometasone furoate treatment group had a significant difference in inhibiting IL-1β, TNF-α and TSLP, the low-dose EPS-L treatment group had a significant difference in inhibiting IL-1 and TNF-α, and the high-dose EPS-H treatment group had a significant difference in inhibiting IL-6, IL-1, TSLP and IL-33.

### Embodiment 30 Influence of Exopolysaccharide of Roseomonas mucosa DL-1 on Filaggrin Expression at Skin Damage of BALB/c Mice with Atopic Dermatitis Induced by MC903

A skin damage tissue of the sacrificed mice in the five groups (the healthy group, the matrix treatment group, the mometasone furoate treatment group and the exopolysaccharide treatment groups) in Embodiment 26 was subjected to immunofluorescent staining by a method as follows:
1) a collected back skin damage tissue of the mice was taken out of −80° C. refrigerator, embedded with OCT, and cut into a 5 mm slice by a freezing microtome; □□
2) the slide was placed in a wet box and rewarmed for 10 minutes; □□
3) the tissue slice was put into PBS and placed in a horizontal shaker to shake at 80 rpm for 5 minutes each time, in a total of 3 times; □□
4) after spreading, the PBS around the tissue was wiped off with a paper towel, and a circle was drawn around the tissue with a Pap Pen;
5) the tissue was dropwise added with a QuickBlock™ blocking buffer for Immunol staining, and incubated at room temperature for 10 minutes; □□
6) the tissue was washed with PBS twice for 5 minutes each time;
7) an FLG Rabbit pAb primary antibody diluted with an immunostaining primary antibody diluent (diluted at 1:1,000, Abclonal, article No. A20011) was dropwise added on a surface of the tissue, and the tissue was incubated in a wet box at 4° C. overnight;
8) the tissue was washed with PBS for 3 times at 80 rpm for 5 minutes each time;
9) an IgG-Alexa Fluor 594 secondary antibody diluted with a secondary antibody diluent (diluted at 1:200, Absin, article No. abs20021) was dropwise added on the surface of the tissue, and the tissue was incubated at room temperature for 60 minutes in the dark; 10) the tissue was washed with PBS for 3 times at 80 rpm for 5 minutes each time; and
10) two drops of DAPI-containing mounting medium were dropwise added on the tissue, the tissue was incubated at room temperature for 5 minutes in the dark to be mounted, and then the mounted tissue was observed under a fluorescent confocal microscope.

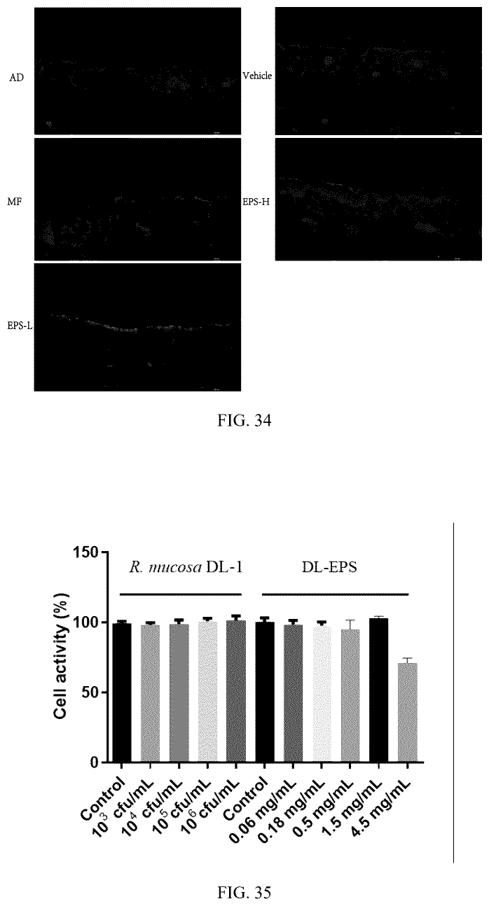

An atopic dermatitis-like skin barrier function damage of the BALB/c mice could be induced by MC903, and filaggrin was one of the structural proteins of epidermis and played a key role in a skin barrier. Results were as shown in FIG. 34. A filaggrin expression in an ear part of the BALB/c mice could be induced to reduce significantly by MC903. Compared with the matrix treatment group, the EPS treatment groups and the mometasone furoate treatment group could all inhibit a decrease of the filaggrin expression induced by MC903, wherein the high-dose exopolysaccharide EPS-H treatment group and the low-dose exopolysaccharide EPS-L treatment group both had a very significant effect, and there were very high fluorescence intensities of filaggrin staining in the two groups.

To sum up, the exopolysaccharide of the Roseomonas mucosa DL-1 provided by the present invention could inhibit the increases of the transcription levels of the inflammatory factors such as TSLP and IL-33 in the HaCaT cells induced by TNF-α and IFN-7. The BALB/c atopic dermatitis animal model induced by MC903 proved a relieving effect of the exopolysaccharide of the Roseomonas mucosa DL-1 on atopic dermatitis-like ear thickening, inflammatory factor aggregation, skin barrier damage, etc. of the mice, so that the exopolysaccharide of the Roseomonas mucosa DL-1 had a certain application prospect in treatment of atopic dermatitis.

### Embodiment 31 Toxicity Assessment of Live Bacterial Formulation of Roseomonas mucosa R. Mucosa DL-1 and Exopolysaccharide DL-EPS of Roseomonas mucosa R. Mucosa DL-1 on Cells

HaCaT cells were inoculated into a 96-well plate at 1×10⁵ cells/well for 3 hours, and then treated with Roseomonas mucosae R. mucosae DL-1 at different final concentrations of 1×10³-1×10⁶ cfu/mL and exopolysaccharides DL-EPS at different concentrations of 0.06-4.5 mg/mL, or added with a blank cell medium as the control, and the cells were incubated in a 5% CO₂ incubator for 24 hours. Each well of the plate was added with 10 L of CCK8 solution, the culture plate was incubated in an incubator for 1-4 hours, and then an absorbance at 450 nm was measured by a microplate reader (Agilent BioTek, SYNERGY/H1). Results were as shown in FIG. 35. 1×10³-1×10⁶ cfu/mL Roseomonas mucosae R. mucosae DL-1 and 0.06-4.5 mg/mL exopolysaccharides DL-EPS had no significant influence on a viability of the HaCaT cells, which indicated that the Roseomonas mucosa DL-1 and the exopolysaccharide DL-EPS thereof were safe in this concentration range.

### Embodiment 32 Influence of Live Bacterial Formulation of Roseomonas mucosa R. Mucosa DL-1 and Exopolysaccharide DL-EPS of Roseomonas mucosa R. Mucosa DL-1 on Reactive Oxygen Species in HaCaT Cells Induced by 50 mJ/Cm² UVB Irradiation

The HaCaT cells were spread in a 6-well culture plate containing 2 mL of 10% FBS-containing DMEM medium at a cell concentration of 3.5×10⁵ cell/well and cultured for 3 hours, and then added with s of the skin symbiotic bacterium R. mucosa DL-1 obtained in Embodiment 2 at concentrations of 1×10⁴ CFU/mL and 1×10⁶ CFU/mL respectively, and exopolysaccharides DL-EPS of the skin symbiotic bacterium R. mucosa DL-1 at concentrations of 0.5 μg/mL and 5 μg/mL. The cells were cultured for a total of 4 hours and then subjected to 50 mJ/cm² UVB irradiation (Sigma irradiation equipment SS-07B (1201B) (280-320 nm) was calibrated according to a Radiometer SOLAR radiometer and a probe PMA2100), and after treatment for 24 hours, reactive oxygen species of the cells were determined.

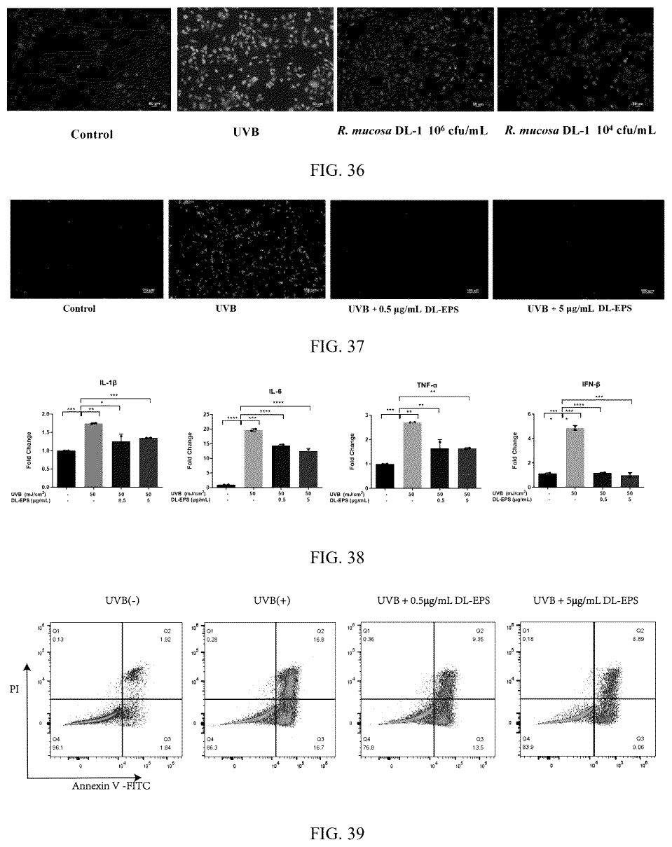

According to a determination method for the reactive oxygen species, DCFH-DA (SLOAR, CA1410) was diluted with a serum-free culture solution at 1:1,000 to reach a final concentration of 10 mol/L. The cell culture solution was removed, and a proper volume of diluted DCFH-DA was added. One well of a 6-well plate was added with 1 mL of diluted DCFH-DA. The cells were incubated in a cell incubator at 37° C. for 20 minutes. The cells were washed with the serum-free cell culture solution for three times to completely remove the DCFH-DA that did not enter the cells. The level of reactive oxygen species in cells of each group was analyzed by a fluorescence microscope. Results were as shown in FIG. 36 and FIG. 37. There was burst of reactive oxygen species in the HaCaT cells after 50 mJ/cm² UVB treatment, the s of the Roseomonas mucosa R. mucosa DL-1 at 1×10⁴ CFU/mL and 1×10⁶ CFU/mL and the exopolysaccharides DL-EPS of the Roseomonas mucosa R. mucosa DL-1 at 0.5 μg/mL and 5 μg/mL could significantly inhibit the reactive oxygen species in the HaCaT cells induced by 50 mJ/cm² UVB irradiation, thereby reducing the level of oxidative stress in the HaCaT cells induced by UVB irradiation.

### Embodiment 33 Influence of Exopolysaccharide DL-EPS of Skin Symbiotic Bacterium R. mucosa DL-1 on Transcription Levels of Main Inflammatory Factors IL-6, IL-1β, TNF-α and IFN-β of HaCaT Cells Induced by 50 mJ/Cm² UVB Irradiation

The HaCaT cells were spread in a 6-well culture plate containing 2 mL of 10% FBS-containing DMEM medium at a cell concentration of 3.5×10⁵ cell/well and cultured for 12 hours, and then added with exopolysaccharides DL-EPS of the skin symbiotic bacterium R. mucosa DL-1 obtained in Embodiment 2 at concentrations of 0.5 μg/mL and g/mL. The cells were cultured for a total of 4 hours and then subjected to 50 mJ/cm² UVB irradiation (Sigma irradiation equipment SS-07B (1201B) (280-320 nm) was calibrated according to a Radiometer SOLAR radiometer and a probe PMA2100), and after irradiation for 4 hours, the same amount of exopolysaccharide DL-EPS was added again for treatment overnight. After UVB treatment for 24 hours, the cells were collected, and a total RNA of the HaCaT cells was extracted with a TRIZOL kit. The total RNA was reversely transcribed to synthesize a cDNA with a reverse transcription kit, and a target mRNA expression level was detected with an SYBR Green kit on an ABI Prism 7900 real-time quantitative PCR instrument. With β-actin as an internal reference, a relative expression quantity of mRNA of a target gene was calculated according to 2^{-ΔΔCt}.

As shown in FIG. 38, the transcription levels of IL-6, IL-1β, TNF-α and IFN-0 of the HaCaT cells could be significantly increased by 50 mJ/cm² UVB irradiation of the HaCaT cells. The exopolysaccharides DL-EPS of the skin symbiotic bacterium R. mucosa DL-1 at concentrations of 0.5 μg/mL and 5 μg/mL could both inhibit increases of the transcription levels of the inflammatory factors IL-6, IL-1 and TNF-α induced by UVB irradiation, and inhibit an increase of the transcription level of the type I interferon IFN-β induced by UVB irradiation.

### Embodiment 34 Influence of Exopolysaccharide DL-EPS of Roseomonas mucosa R. Mucosa DL-1 on Apoptosis of HaCaT Cells Induced by 50 mJ/Cm² UVB Irradiation

A cell experiment was set as Embodiment 32, and apoptosis of the HaCaT cells after 50 mJ/cm² UVB treatment for 24 hours was analyzed. After treatment with an apoptosis detection kit (Biolegend, 640914) according to recommended staining steps, analysis was made by a flow cytometer. As shown in FIG. 39, 50 mJ/cm² UVB irradiation of the HaCaT cells could cause apoptosis of a large number of HaCaT cells, and 0.5 μg/mL and g/mL exopolysaccharides DL-EPS could both significantly inhibit the apoptosis of the HaCaT cells induced by UVB irradiation.

### Embodiment 35 Influences of live bacterial formulation of Roseomonas mucosa R. mucosa DL-1 and exopolysaccharide DL-EPS of Roseomonas mucosa R. mucosa DL-1 on pathologies of skin damage visible to naked eye and skin damage under microscope on back part of C57BL/6 mice induced by 430 mJ/cm² UVB irradiation

Animal experimental design: a total of 28 C57BL/6J mice, aged 7 weeks, were randomly divided into 7 groups, and 24 hours after shaving back parts of the mice in all groups, the mice were respectively treated with a drug. The seven groups were a healthy control group (no treatment, NC), a UVB model group (UVB), a positive control group (hydrocortisone 50 mg/4 cm²/day, UVB+Hydr), a matrix group (100% propylene glycol, UVB+Vehicle), a high-dose group (1 mg/4 cm²/day, UVB+EPS-H), a low-dose group (0.5 mg/4 cm²/day, UVB+EPS-L), a viable bacterial treatment group (10⁹ cfu/4 cm²/day, UVB+DL-1) respectively. After treatment for 3 days, the back part was subjected to 430 mJ/cm² UVB irradiation, and the drug was continuously administered until UVB irradiation for 48 hours.

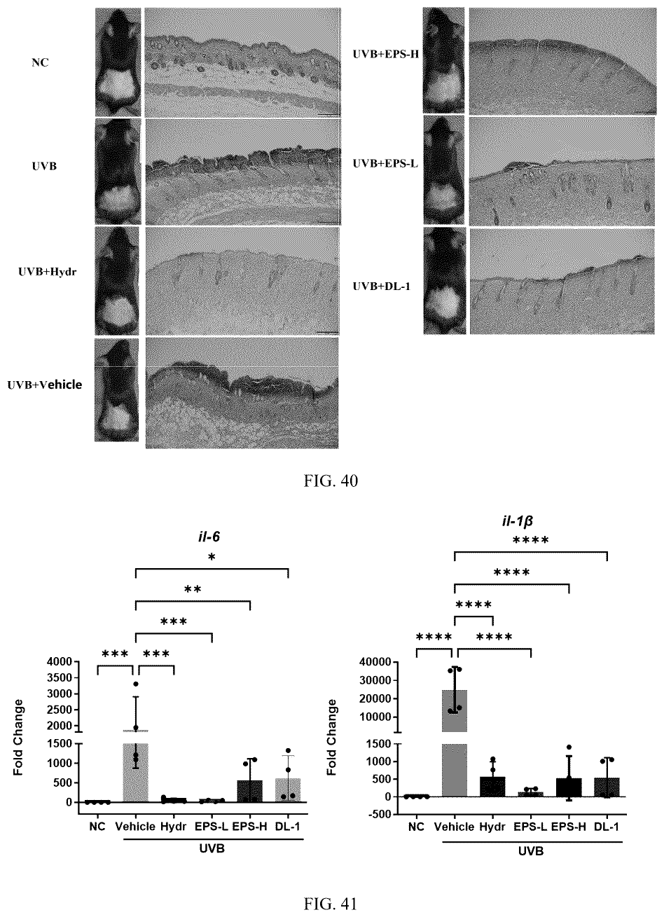

Statistical analysis method: the mice were sacrificed after irradiation for 48 hours, and erythra on the back part of the mice was observed. Experimental results were as shown in FIG. 40. There were skin erythema, edema, exudation and scabbing in the UVB model group, forming a diffuse skin damage. Compared with the matrix group, the hydrocortisone of the positive group could significantly relieving skin erythema, edema, exudation and scabbing induced by acute UVB irradiation. In the experimental groups, treatments with the 0.5 mg/4 cm²/day DL-EPS and the live bacterial formulation of the Roseomonas mucosa DL-1 on the mice could relieve skin erythema, edema, exudation and scabbing induced by acute UVB irradiation, with only a local skin damage, thereby having broad application prospects in skin diseases induced by UVB.

### Embodiment 36 Influences of live bacterial formulation of Roseomonas mucosa R. mucosa DL-1 and exopolysaccharide DL-EPS of Roseomonas mucosa R. mucosa DL-1 on transcriptions of local main inflammatory factors IL-6, IL-1β and TNF-α at skin damage on back part of C57BL/6 mice induced by 430 mJ/cm² UVB irradiation

A skin damage of the sacrificed mice in Embodiment 35 was collected and fully ground by a tissue grinder, and then a total RNA of HaCaT cells was extracted with a TRIZOL kit. The total RNA was reversely transcribed to synthesize a cDNA with a reverse transcription kit, and a target mRNA expression level was detected with an SYBR Green kit on an ABI Prism 7900 real-time quantitative PCR instrument. With β-actin as an internal reference, a relative expression quantity of mRNA of a target gene was calculated according to 2^{-ΔΔCt}.

Results were as shown in FIG. 41. Transcription levels of main inflammatory factors IL-6 and IL-1β at the skin damage could be induced to increase significantly by acute UVB irradiation. Compared with the matrix treatment group, treatments with the hydrocortisone, the high-dose DL-EPS, the low-dose DL-EPS and the live bacterial formulation of the Roseomonas mucosa R. mucosa DL-1 could all inhibit increases of the transcription levels of the main inflammatory factors IL-6 and IL-1β at the skin damage induced by acute UVB irradiation. The groups all had a statistical significance, wherein the low-dose DL-EPS treatment group had the most significant effect of inhibiting the increases of the transcription levels of the acute inflammatory factors induced by UVB.

### Embodiment 37 Influences of live bacterial formulation of Roseomonas mucosa R. mucosa DL-1 and exopolysaccharide DL-EPS of Roseomonas mucosa R. mucosa DL-1 on apoptosis of local epithelial keratinocytes at skin damage on back part of C57BL/6 mice induced by 430 mJ/cm² UVB irradiation

An immunofluorescence staining method for a skin tissue of the mice was as follows: 1) a collected back skin damage tissue of the mice was taken out of −80° C. refrigerator, embedded with OCT, and cut into a 5 mm slice by a freezing microtome; □□
2) the slide was placed in a wet box and rewarmed for 10 minutes; □□
3) the tissue slice was put into PBS and placed in a horizontal shaker to shake at 80 rpm for 5 minutes each time, in a total of 3 times; Q D
4) after spreading, the PBS around the tissue was wiped off with a paper towel, and a circle was drawn around the tissue with a Pap Pen;
5) the tissue was dropwise added with a QuickBlock™ blocking buffer for Immunol staining, and incubated at room temperature for 10 minutes;
6) the tissue was washed with PBS twice for 5 minutes each time;
7) an anti-pancytokeratin primary antibody diluted with an immunostaining primary antibody diluent (diluted at 1:100, abeam, article No. ab8068) was dropwise added on a surface of the tissue, and the tissue was incubated in a wet box at 4° C. overnight;
8) the tissue was washed with PBS for 3 times at 80 rpm for 5 minutes each time;
9) an Alexa Fluor 488-conjugated goat ant-rabbite secondary antibody diluted with a secondary antibody diluent (diluted at 1:200, vector lab, article No. DI-1488-1.5) was dropwise added on the surface of the tissue, and the tissue was incubated at room temperature for 60 minutes in the dark; 10) the tissue was washed with PBS for 3 times at 80 rpm for 5 minutes each time;
10) a propidium iodide (PI)/RNase staining solution (Cell Signaling Technology, 4087S) was dropwise added on the tissue, and the tissue was incubated at room temperature for 15 minutes in the dark;
11) the tissue was washed with PBS for 3 times at 80 rpm for 5 minutes each time; and
12) two drops of DAPI-containing mounting medium were dropwise added on the tissue, the tissue was incubated at room temperature for 5 minutes in the dark to be mounted, and then the mounted tissue was observed under a fluorescent confocal microscope.

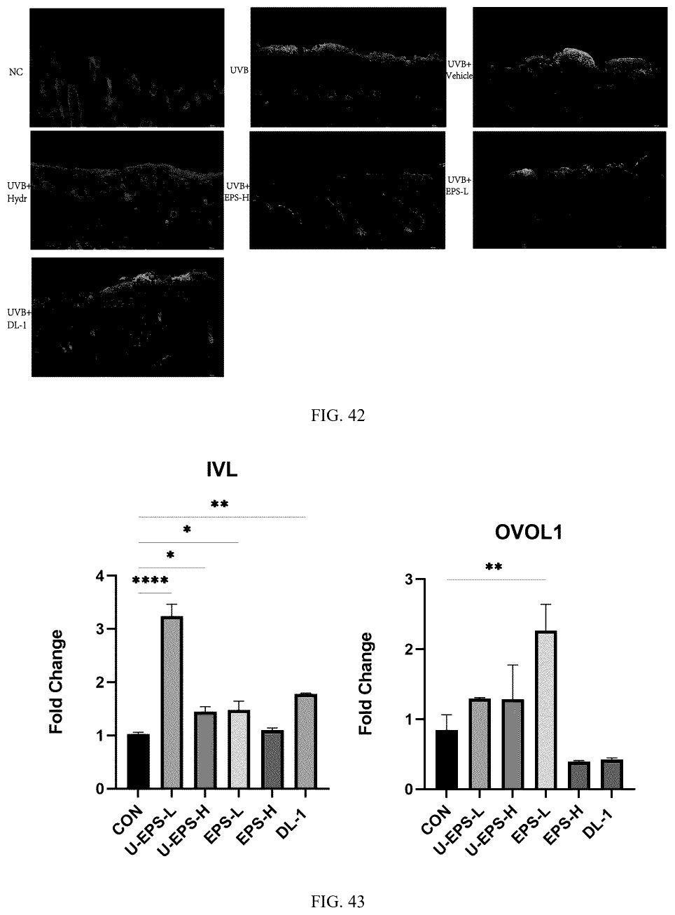

Results were as shown in FIG. 42. The UVB model group and the matrix group both showed typical apoptosis of the epidermal keratinocytes after UVB irradiation with a total amount of 430 mJ/cm², and the positive hydrocortisone treatment group and the high-dose exopolysaccharide treatment group could both significantly relieve the apoptosis of the keratinocytes induced by UVB. The low-dose exopolysaccharide treatment group and the treatment group of the live bacterial formulation of the Roseomonas mucosa DL-1 had a trend of improving the apoptosis of the keratinocytes induced by UVB.

To sum up, the live bacterial formulation of the skin symbiotic bacterium Roseomonas mucosa and the exopolysaccharide of the skin symbiotic bacterium Roseomonas mucosa provided by the present invention were used to treat the HaCaT cells after UVB irradiation and the acute damage on the back part of the C57/BL6 mice induced by UVB. The Roseomonas mucosa DL-1 and the exopolysaccharide thereof had a protective effect on the apoptosis of the keratinocytes induced by UVB in the cell model and the mouse model, could inhibit the increases of the transcription levels of the local inflammatory factors at the skin damage induced by UVB, and could reduce the apoptosis of the HaCaT cells induced by UVB irradiation. The present invention could provide a certain scientific basis for the development of a drug for antagonizing a light damage.

### Embodiment 38 Experiment of Roseomonas mucosa DL-1 and Exopolysaccharide Thereof in Promoting Expression of Gene Related to Barrier Repair of HaCaT

The HaCaT cells were spread in a 6-well culture plate containing 2 mL of 10% FBS-containing DMEM medium at a cell concentration of 1×10⁵ cell/well and cultured overnight until the cells were adhered. The cells were respectively added with 1×10⁶ CFU/mL live bacterial formulation of the Roseomonas mucosa DL-1, and crude exopolysaccharides DL-U-EPS and pure exopolysaccharides EPS at concentrations of 0.5 g/mL (U-EPS-L, EPS-L) and 5 μg/mL (U-EPS-H, EPS-H) respectively obtained in Embodiment 1, and the control group was added with the equal volume of medium. After being cultured for 24 hours, for the cells collected above, a total RNA of the HaCaT cells was extracted with a TRIZOL kit. The total RNA was reversely transcribed to synthesize a cDNA with a reverse transcription kit, and mRNA expression levels of IVL and OVOL1 were detected with an SYBR Green kit on an ABI Prism 7900 real-time quantitative PCR instrument. With β-actin as an internal reference, treatment groups added with the equal volume of medium were respectively used as control (relative expression fold of gene F=1), and a relative expression quantity of mRNA of a target gene was calculated according to 2^{-ΔΔCt}.

As shown in FIG. 43, the Roseomonas mucosa DL-1 and the exopolysaccharide thereof had an effect of up-regulating expression of a skin barrier repair-related factor involucrin gene IVL and an OVO-like transcription factor 1 gene OVOL1, and the gene expression was up-regulated by 1.41-3.16 times, wherein the low-dose crude exopolysaccharide DL-U-EPS had the best up-regulation effect on IVL, P<0.0001; and the low-dose pure exopolysaccharide EPS had the best up-regulation effect on OVOL1, P<0.01. It was indicated that the Roseomonas mucosa DL-1 and the exopolysaccharide thereof had an effect of promoting skin barrier repair.

### Embodiment 39 Experiment of Roseomonas mucosa DL-1 and Exopolysaccharide Thereof in Up-Regulating Expression of Gene Related to Moisturization of HaCaT

The HaCaT cells were spread in a 6-well culture plate containing 2 mL of 10% FBS-containing DMEM medium at a cell concentration of 1×10⁵ cell/well and cultured overnight until the cells were adhered. The cells were respectively added with 1×10⁶ CFU/mL live bacterial formulation of the Roseomonas mucosa DL-1, and crude exopolysaccharides DL-U-EPS and pure exopolysaccharides EPS at concentrations of 0.5 g/mL (U-EPS-L, EPS-L) and 5 μg/mL (U-EPS-H, EPS-H) respectively obtained in Embodiment 1, and the control group was added with the equal volume of medium. After being cultured for 24 hours, for the cells collected above, a total RNA of the HaCaT cells was extracted with a TRIZOL kit. The total RNA was reversely transcribed to synthesize a cDNA with a reverse transcription kit, and the mRNA expression level of AQP3 was detected with an SYBR Green kit on an ABI Prism 7900 real-time quantitative PCR instrument. With β-actin as an internal reference, treatment groups added with the equal volume of medium were respectively used as control (relative expression fold of gene F=1), and a relative expression quantity of mRNA of a target gene was calculated according to 2^{-ΔΔCt}.

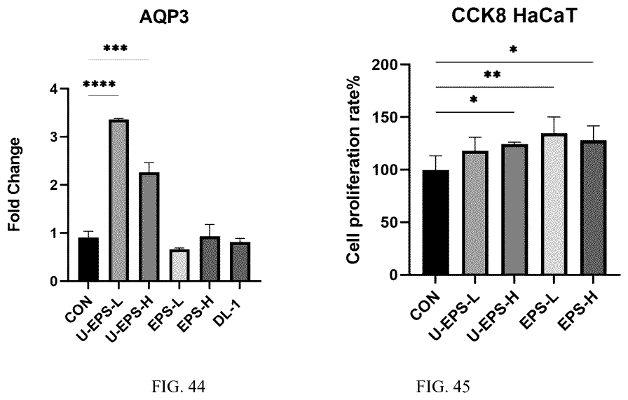

As shown in FIG. 44, the Roseomonas mucosa DL-1 and the exopolysaccharide thereof had an effect of up-regulating the aquaporin 3 gene AQP3 related to moisturization, and the gene expression was up-regulated by 2.49-3.70 times, wherein the low-dose and high-dose crude exopolysaccharides could both significantly up-regulate the AQP3, with P values of P<0.0001 and P<0.001 respectively. It was indicated that the Roseomonas mucosa DL-1 and the exopolysaccharide thereof had an effect of promoting skin moisturization.

### Embodiment 40 Experiment of Roseomonas mucosa DL-1 and Exopolysaccharide Thereof in Promoting Proliferation of HaCaT Cells

The HaCaT cells were spread in a 96-well culture plate containing 100 μL of 10% FBS-containing DMEM medium at a cell concentration of 3×10⁴ cell/well and cultured overnight until the cells were adhered. The cells were respectively added with crude exopolysaccharides DL-U-EPS and pure exopolysaccharides EPS of the Roseomonas mucosa DL-1 at concentrations of 0.5 μg/mL (U-EPS-L, EPS-L) and 5 μg/mL (U-EPS-H, EPS-H) respectively obtained in Embodiment 1, and the control group was added with the equal volume of medium. After being cultured for 24 hours, Each well was added with 10 μl of CCK-8 solution and cultured for 1 hour, and then an absorbance A at 450 nm was detected, according to a calculation formula: cell proliferation rate=(experimental group A−control group A)/control group A*100%.

As shown in FIG. 45, the exopolysaccharide of the Roseomonas mucosa had an effect of promoting on proliferation of HaCaT keratinocytes, at a proliferation rate of 13.8%-48.33%.

### Embodiment 41 Experiment of Roseomonas mucosa DL-1 and Exopolysaccharide Thereof in Regulating Extracellular Matrix/Anti-Oxidation/Apoptosis Inhibition/Cell Growth Factor/Apoptosis-Related Gene Expression of HFF Cells with Oxidative Damage

The HFF cells were spread in a 6-well culture plate containing 2 mL of 15% FBS-containing DMEM medium at a cell concentration of 3×10⁵ cell/well and cultured overnight until the cells were adhered. Each well was added with H₂O₂ at a final concentration of 200 μM for stimulation, and then allowed to stand at 37° C. for 1 hour. The original medium was discarded, and the cells were washed with PBS twice, and added with 2 mL of new medium. The cells were respectively added with 1×10⁶ CFU/mL live bacterial formulation of the Roseomonas mucosa DL-1, and crude exopolysaccharides U-EPS and pure exopolysaccharides at concentrations of 0.5 μg/mL (U-EPS-L, EPS-L) and 5 μg/mL (U-EPS-H, EPS-H) respectively obtained in Embodiment 1, and the control group was added with the equal volume of medium. After being cultured for 4 hours, for the cells collected above, a total RNA of the HFF cells was extracted with a TRIZOL kit. The total RNA was reversely transcribed to synthesize a cDNA with a reverse transcription kit, and mRNA expression levels of extracellular matrix synthesis-related genes (SPTSSA, MKX and SMAD3), antioxidant gene NRF2, apoptosis inhibitory gene (BCL-2), cell growth factor-related genes (FGF1, FGF2 and HGF) were detected with an SYBR Green kit on an ABI Prism 7900 real-time quantitative PCR instrument. With β-actin as an internal reference, treatment groups added with the equal volume of medium were respectively used as control (relative expression fold of gene F=1), and a relative expression quantity of mRNA of a target gene was calculated according to 2^{-ΔΔCt}.

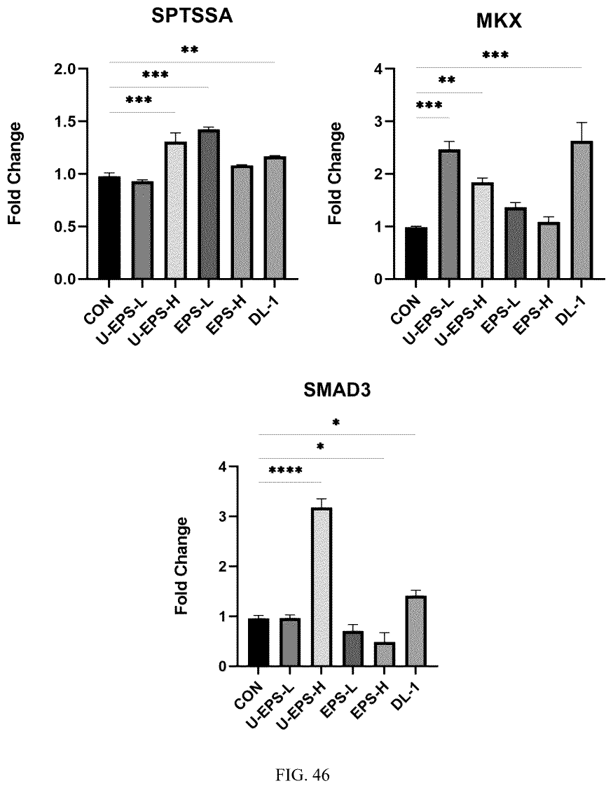

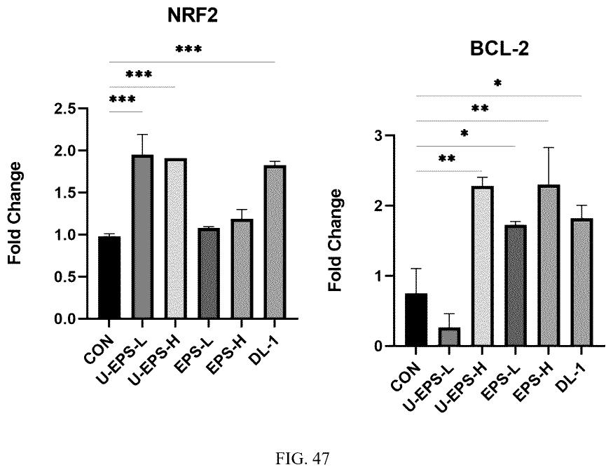

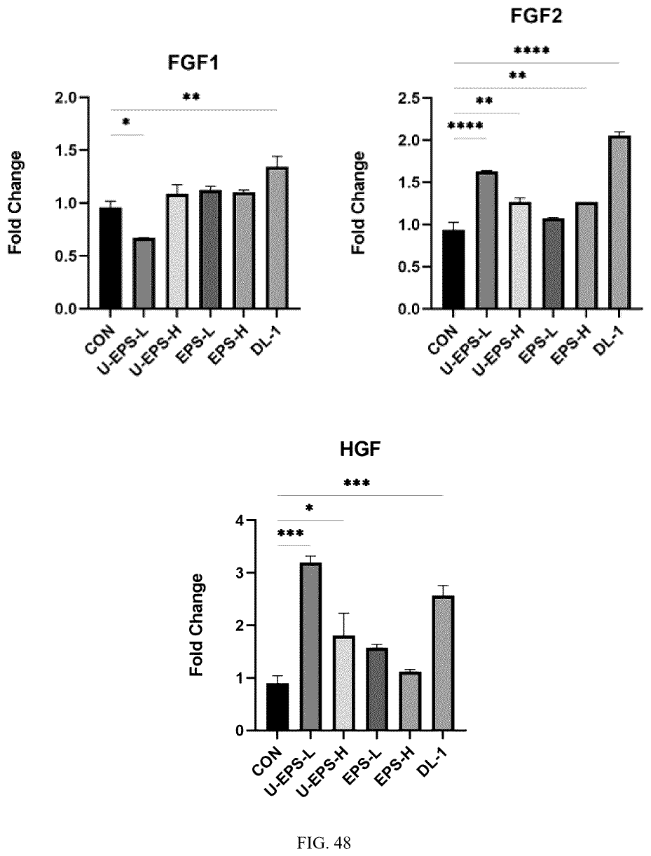

As shown in FIG. 46 to FIG. 48, the Roseomonas mucosa DL-1 and the exopolysaccharide thereof could up-regulate the mRNA expression of a serine palmitoyltransferase gene SPTSSA related to extracellular matrix synthesis of the HFF cells, wherein the high-dose crude exopolysaccharide treatment group (U-EPS-L), the low-dose pure exopolysaccharide treatment group (EPS-L) and the Roseomonas mucosa DL-1 treatment group had a significant difference. The Roseomonas mucosa DL-1 and the exopolysaccharide thereof could up-regulate the mRNA expression of a Mohawk protein gene MKX, wherein the high-dose and low-dose crude exopolysaccharide treatment groups and the Roseomonas mucosa DL-1 treatment group had a significant difference. The Roseomonas mucosa DL-1 and the crude exopolysaccharide thereof could up-regulate the mRNA expression of a signal transduction protein gene SMAD3, wherein the high-dose crude exopolysaccharide treatment group and the Roseomonas mucosa DL-1 treatment group had a significant difference. The Roseomonas mucosa DL-1 and the exopolysaccharide thereof could up-regulate the mRNA expression of an antioxidation-related nuclear factor E2-related factor 2 gene NRF2, and the gene expression of the above gene was up-regulated by 1.20-3.54 times, wherein the crude exopolysaccharide treatment groups and the Roseomonas mucosa DL-1 treatment group had a significant difference. The Roseomonas mucosa DL-1 and the exopolysaccharide thereof could up-regulate the mRNA expression of an apoptosis inhibition-related B lymphoblastoma-2 gene BCL-2, wherein the high-dose crude exopolysaccharide treatment group, the high-dose and low-dose pure exopolysaccharide treatment groups and the Roseomonas mucosa DL-1 treatment group had a significant difference. The Roseomonas mucosa DL-1 and the crude exopolysaccharide thereof could up-regulate a cell growth factor gene FGF1, wherein the Roseomonas mucosa DL-1 treatment group had a significant difference. The Roseomonas mucosa DL-1 and the exopolysaccharide thereof could up-regulate the mRNA expression of a cell growth factor gene FGF2, wherein the crude exopolysaccharide treatment groups, the high-dose pure exopolysaccharide treatment group and the Roseomonas mucosa DL-1 treatment group had a significant difference. The Roseomonas mucosa DL-1 and the exopolysaccharide thereof could up-regulate the mRNA expression of a hepatocyte growth factor gene HGF, wherein the crude exopolysaccharide treatment groups and the Roseomonas mucosa DL-1 treatment group had a significant difference (* denotes that P<0.05, ** denotes that P<0.01, *** denotes that P<0.001, and **** denotes that P<0.0001). Influences of the Roseomonas mucosa DL-1 and the exopolysaccharide thereof on the above cell aging-related indexes (extracellular matrix, antioxidation, apoptosis inhibition, cell growth factor and apoptosis-related gene expression) were determined, and results showed that the Roseomonas mucosa DL-1 and the exopolysaccharide thereof had an anti-aging effect.

### Embodiment 42 Experiment of Roseomonas mucosa DL-1 and Exopolysaccharide Thereof in Up-Regulating Expression of Gene Related to Antibacterial Peptide of HaCaT Cells

The HaCaT cells were spread in a 6-well culture plate containing 2 mL of 10% FBS-containing DMEM medium at a cell concentration of 1×10⁵ cell/well and cultured overnight until the cells were adhered. The cells were respectively added with crude exopolysaccharides DL-U-EPS and pure exopolysaccharides EPS of the Roseomonas mucosa DL-1 at concentrations of 0.5 μg/mL (U-EPS-L, EPS-L) and 5 μg/mL (U-EPS-H, EPS-H) respectively obtained in Embodiment 1, and the control group was added with the equal volume of medium. 2 hours later, each well was added with an LPS solution at a final concentration of 200 ng/ml. After being cultured for 24 hours, for the cells collected above, a total RNA of the HaCaT cells was extracted with a TRIZOL kit. The total RNA was reversely transcribed to synthesize a cDNA with a reverse transcription kit, and mRNA expression levels of S100A7, DEFB4 and LL37 were detected with an SYBR Green kit on an ABI Prism 7900 real-time quantitative PCR instrument. With β-actin as an internal reference, treatment groups added with the equal volume of medium were respectively used as control (relative expression fold of gene F=1), and a relative expression quantity of mRNA of a target gene was calculated according to 2^{-ΔΔCt}.

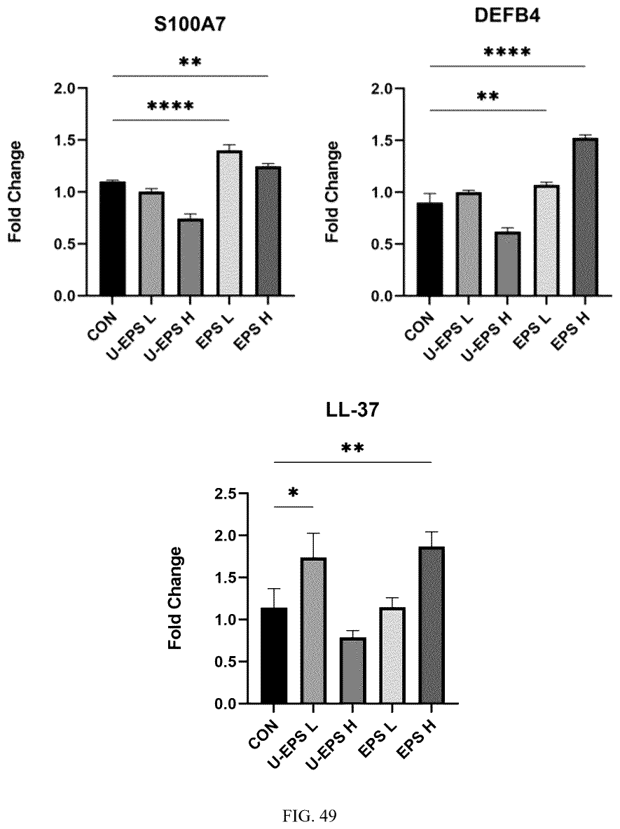

As shown in FIG. 49, the exopolysaccharide of the Roseomonas mucosa DL-1 had an effect of promoting expression of antibacterial peptide-related genes, such as psoriasin S100A7, β-defensin 4 DEFB4 and cathelicidin family anti-bacterial peptide LL-37, and the gene expression was up-regulated by 1.071-1.867 times, wherein the pure exopolysaccharides of the Roseomonas mucosa DL-1 had the most significant effect (* denotes that P<0.05, ** denotes that P<0.01, *** denotes that P<0.001, and **** P<0.0001). It was indicated that the exopolysaccharide of the Roseomonas mucosa DL-1 had an effect of enhancing a skin antimicrobial defense.

### Embodiment 43 Experiment of Roseomonas mucosa DL-1 and Exopolysaccharide Thereof in Regulating Expression of Gene Related to Renewal of Aged Stratum Corneum of HaCaT Cells

The HaCaT cells were spread in a 6-well culture plate containing 2 mL of 10% FBS-containing DMEM medium at a cell concentration of 1×10¹ cell/well and cultured overnight until the cells were adhered. The cells were respectively added with 1×10⁶ CFU/mL live bacterial formulation of the Roseomonas mucosa DL-1, and crude exopolysaccharides DL-U-EPS and pure exopolysaccharides EPS at concentrations of 0.5 g/mL (U-EPS-L, EPS-L) and 5 μg/mL (U-EPS-H, EPS-H) respectively obtained in Embodiment 1, and the control group was added with the equal volume of medium. An ultraviolet photoaging experiment with a total amount of 40 mj/cm² was carried out on the cells in the wells. After being cultured for 4 hours, for the cells collected above, a total RNA of the HaCaT cells was extracted with a TRIZOL kit. The total RNA was reversely transcribed to synthesize a cDNA with a reverse transcription kit, and mRNA expression levels of KLK5 and CD44 were detected with an SYBR Green kit on an ABI Prism 7900 real-time quantitative PCR instrument. With R-actin as an internal reference, treatment groups added with the equal volume of medium were respectively used as control (relative expression fold of gene F=1), and a relative expression quantity of mRNA of a target gene was calculated according to 2^{-ΔΔCt}.

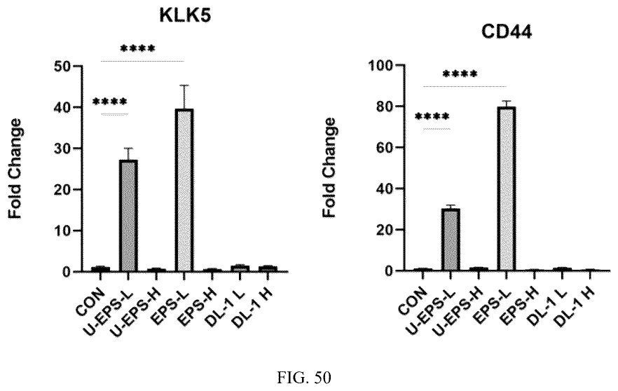

As shown in FIG. 50, the Roseomonas mucosa DL-1 and the exopolysaccharide thereof could up-regulate expression of a desmoplakin degradation-related gene kallikrein 5 gene KLK5 and a transmembrane adhesive glycoprotein CD44, and the gene expression was up-regulated by 23.60-73.12 times, wherein the low-dose crude exopolysaccharide treatment group and the (low-dose and high-dose) pure exopolysaccharide treatment groups EPS all had a significant effect of up-regulating KLK5 and CD44, with P values less than 0.0001, and had a significant difference. Therefore, the Roseomonas mucosa DL-1 and the exopolysaccharide thereof could promote desmoplakin degradation among aged stratum corneocytes to exfoliate the aged stratum corneocytes, and regulate keratinocyte differentiation to renew the keratinocytes, thereby having an anti-aging effect.

What is claimed is:

1. A method for preventing and treating a disease comprising a step of administering a drug to a subject in need, wherein the drug is an exopolysaccharide of the Roseomonas mucosa or a live bacterial formulation, the disease is selected from the group consisting of an inflammation-related disease, a T/B cell over-activation disease, a cutaneous lupus erythematosus skin damage, psoriasis, an atopic dermatitis and a UVB-induced skin damage, wherein the Roseomonas mucosa is Roseomonas mucosa DL-1 deposited in China General Microbiological Culture Collection Center with a deposition number as CGMCC No. 25967; the live bacterial formulation contains the Roseomonas mucosa, and the exopolysaccharide of the Roseomonas mucosa, is obtained by sterilization, deproteinization, precipitation with absolute ethanol, and separation and purification with DEAE centrifugal exchange resin of a fermentation broth containing the Roseomonas mucosa.

2. The method according to claim 1, wherein a count of live Roseomonas mucosa in the live bacterial formulation is 1×10⁴-9×10¹¹ CFU/mL.

3. The method according to claim 1, wherein the Roseomonas mucosa is inoculated in an R2A medium to activate at 25-37° C. for 12-24 hours, the activated Roseomonas mucosa is transferred to a fermentation medium to culture at 30-37° C. for 24-36 hours, and then centrifuged at 4,000-9,000 rpm for 5-10 minutes to obtain a Roseomonas mucosa, and the Roseomonas mucosa is added into a vehicle to obtain the live bacterial formulation.

4. The method according to claim 1, wherein a weight-average molecular weight of the exopolysaccharide of the Roseomonas mucosa is 3,000-3,500 Da.

5. The method according to claim 1, wherein the exopolysaccharide of the Roseomonas mucosa is composed of arabinose, rhamnose, galactose, glucose, xylose, mannose, ribose, galacturonic acid, glucuronic acid, mannuronic acid and guluronic acid.

6. The method according to claim 1, wherein the exopolysaccharide of the Roseomonas mucosa inhibits differentiation of B cells into plasmablasts and antibody secreting cells induced in vitro, the exopolysaccharide of the Roseomonas mucosa inhibits activation of T cells into Tfh cells induced by anti-CD28 in vitro.

7. The method according to claim 1, wherein the cutaneous lupus erythematosus skin damage comprises one or more of acute cutaneous lupus erythematosus, subacute cutaneous lupus erythematosus, chronic cutaneous lupus erythematosus and subtypes thereof, and the live bacterial formulation or the exopolysaccharide of the Roseomonas mucosa regulates an immune microenvironment at the skin damage, thereby reducing deposition of IgG immune complexes.

8. The method according to claim 1, wherein the live bacterial formulation of the Roseomonas mucosa relieves a psoriasis-like skin damage on a back part of a BALB/c mouse induced by Imiquimod, inhibits an increase of a spleen index of the BALB/c mouse with psoriasis induced by imiquimod, and relieves epidermal thickening of the BALB/c mouse induced by imiquimod.

9. The method according to claim 1, wherein the exopolysaccharide of the Roseomonas mucosa inhibits proliferation of HaCaT cells induced by M5 and inhibits an increase of a the mRNA expression of an inflammatory factor of the HaCaT cells induced by M5, and the M5 is a combination of TNF-α, oncostatin-M, IL-17A, IL-1α and IL-22 relieves a psoriasis-like skin damage on a back part of a BALB/c mouse induced by imiquimod, inhibits an increase of a spleen index of a BALB/c mouse with psoriasis induced by imiquimod and reduces epidermal thickness of a BALB/c mouse induced by imiquimod.

10. The method according to claim 1, wherein the exopolysaccharide of the Roseomonas mucosa inhibits the mRNA expression the mRNA expression and secretion of an inflammatory factor of HaCaT cells induced by TNF-α and IFN-7, and the inflammatory factor is any one or a combination of several of IL-1β, TNF-α, IL-6, TSLP and IL-33.

11. The method according to claim 1, wherein the exopolysaccharide of the Roseomonas mucosa relieves ear swelling and scales and ear mastocytosis of a BALB/c mouse induced by MC903, reduces ear thickness and epidermal thickness of a BALB/c mouse with atopic dermatitis induced by MC903, and relieves mast cell infiltration of the BALB/c mouse induced by MC903.

12. The method according to claim 1, wherein the exopolysaccharide of the Roseomonas mucosa inhibits an increase of the mRNA expression the mRNA expression of one or more of inflammatory factors IL-1β, TNF-α, IL-6, TSLP and IL-33 at a skin damage of a BALB/c mouse induced by MC903, and inhibits a decrease of a filaggrin expression at a skin damage of a BALB/c mouse induced by MC903.

13. The method according to claim 1, wherein when the live bacterial formulation is used to preventing and treating a UVB-induced skin damage a count of the live Roseomonas mucosa in the live bacterial formulation is 1×10⁶-1×10⁹ CFU/mL.

14. The method according to claim 1, wherein the live bacterial formulation of the Roseomonas mucosa reduces the level of oxidative stress and apoptosis of HaCaT cells induced by UVB irradiation improves skin erythema, edema, exudation and scabbing on a back part of a C57BL/6 mouse induced by UVB irradiation, inhibits apoptosis of keratinocytes at a skin damage on the back part induced by UVB irradiation, and reduces the mRNA expression the mRNA expression of an inflammatory factor at the skin damage.

15. The method according to claim 1, wherein the exopolysaccharide of the Roseomonas mucosa reduces oxidative stress and apoptosis of HaCaT cells induced by UVB irradiation, reduces the mRNA expression the mRNA expression of an inflammatory factor of the HaCaT cells induced by UVB irradiation, inhibits an increase of the mRNA expression of a type I Interferon IFN-β induced by UVB irradiation, improves skin erythema, edema, exudation and scabbing on a back part of a C57BL/6 mouse induced by UVB irradiation, inhibits apoptosis of keratinocytes at a skin damage on the back part induced by UVB irradiation, and reduces the mRNA expression of an inflammatory factor at the skin damage.

16. A method for improving a skin condition comprising a step of administering a drug to a subject in need, wherein the drug is an exopolysaccharide of the Roseomonas mucosa or a live bacterial formulation, the skin condition comprises at least one of improvement of skin barrier, moisturization, enhancement of skin antibacterial ability, renewal of aged stratum corneum and anti-aging; wherein the Roseomonas mucosa is Roseomonas mucosa DL-1 deposited in China General Microbiological Culture Collection Center with a deposition number as CGMCC No. 25967; the live bacterial formulation contains the Roseomonas mucosa, and the exopolysaccharide of the Roseomonas mucosa, is obtained by sterilization, deproteinization, precipitation with absolute ethanol, and separation and purification with DEAE centrifugal exchange resin of a fermentation broth containing the Roseomonas mucosa.

17. The method according to claim 16, wherein, in application, the exopolysaccharide of the Roseomonas mucosa is applied in a form of a crude product or a pure product, a weight-average molecular weight of a crude exopolysaccharide of the Roseomonas mucosa is 1,100-2,000 kDa; and a weight-average molecular weight of a pure exopolysaccharide of the Roseomonas mucosa is 3,000-3,500 Da.

18. The method according to claim 16, wherein the improvement of skin barrier refers to restoring a cell viability cell viability and/or positively regulating expression of a barrier repair-related gene, and promoting proliferation of HaCaT cells; and the barrier repair-related gene comprises at least one of IVL and OVOL1; the moisturization refers to positively regulating expression of a moisturization-related gene AQP3; wherein the enhancement of skin antimicrobial defense comprises up-regulating expression of an antibacterial peptide-related gene, and the antibacterial peptide-related gene comprises at least one of a psoriasin S100A7, a β-defensin 4 DEF4 and a cathelicidin family antibacterial peptide LL-37; the anti-aging comprises at least one of the following i)-iv):
i) positively regulating expression of an extracellular matrix-related gene, wherein the extracellular matrix-related gene comprises at least one of a serine palmitoyltransferase gene SPTSSA, a Mohawk protein gene MKX and a signal transduction protein gene SMAD3;
ii) positively regulating expression of an apoptosis inhibition-related gene BCL-2;
iii) positively regulating expression of a cell antioxidation-related gene NRF2; and
iv) positively regulating expression of a cell growth factor-related gene, wherein the cell growth factor-related gene comprises at least one of FGF1, FGF2 and HGF; the renewal of aged stratum corneum comprises up-regulating expression of a desmoplakin degradation-related gene KLK5 and/or up-regulating expression of a keratinocyte differentiation-related gene CD44.

19. The method according to claim 16, wherein a form of the product is any one of cream, emulsion, oil, water, gel, powder and a freeze-dried product.

20. An exopolysaccharide of the Roseomonas mucosa, wherein the exopolysaccharide of the Roseomonas mucosa is obtained by sterilization, deproteinization, precipitation with absolute ethanol, and separation and purification with DEAE centrifugal exchange resin of a fermentation broth containing Roseomonas mucosa, wherein the Roseomonas mucosa is Roseomonas mucosa DL-1 deposited in China General Microbiological Culture Collection Center with a deposition number as CGMCC No. 25967.
